(19) United States
(12) Patent Application Publication
(10) Pub. No.: US 2026/0096776 A1
(43) Pub. Date: Apr. 9, 2026

(54) ELECTRONIC DEVICE FOR PROVIDING MUSCULAR EXERCISE GUIDANCE AND OPERATING METHOD THEREOF

(71) Applicant: Samsung Electronics Co., Ltd., Suwon-si (KR)

(72) Inventors: Chungdo KIM, Suwon-si (KR); Jieung JUN, Suwon-si (KR); Minkyung HWANG, Suwon-si (KR)

(21) Appl. No.: 19/332,820

(22) Filed: Sep. 18, 2025

Related U.S. Application Data
(63) Continuation of application No. PCT/KR2025/013174, filed on Aug. 28, 2025.

(30) Foreign Application Priority Data
Oct. 7, 2024 (KR) 10-2024-0136088
Nov. 11, 2024 (KR) 10-2024-0159412

(51) Int. Cl. A61B 5/00; A61B 5/024; A61B 5/026; A61B 5/0295; A61B 5/22; A61B 5/256

(52) U.S. Cl. CPC A61B 5/681; A61B 5/02438; A61B 5/0261; A61B 5/0295; A61B 5/222; A61B 5/225; A61B 5/256; A61B 5/7275; A61B 5/7475

(57) ABSTRACT

A wearable electronic device is provided. The wearable electronic device includes a biosensor including at least one light emitter and at least one light receiver, the biosensor being configured to sense blood flow of a user wearing the wearable electronic device, communication circuitry, an output interface, memory, comprising one or more storage media, storing instructions, and one or more processors communicatively coupled to the biosensor, the communication circuitry, the output interface, and the memory, wherein the instructions, when executed by the one or more processors individually or collectively, cause the wearable electronic device to obtain biometric data related to the blood flow of the user via the biosensor while an application related to a muscular exercise is executed, identify a grip strength change based on a change in the blood flow observed in the biometric data resulting from the muscular exercise performed by the user, create exercise information related to the muscular exercise based on the grip strength change, and provide the user with the exercise information via at least one of the communication circuitry or the output interface.

### CROSS-REFERENCE TO RELATED APPLICATION(S)

This application is a continuation application, claiming priority under 35 U.S.C. § 365(c), of an International application No. PCT/KR2025/013174, filed on Aug. 28, 2025, which is based on and claims the benefit of a Korean patent application number 10-2024-0136088, filed on Oct. 7, 2024, in the Korean Intellectual Property Office, and of a Korean patent application number 10-2024-0159412, filed on Nov. 11, 2024, in the Korean Intellectual Property Office, the disclosure of each of which is incorporated by reference herein in its entirety.

### TECHNICAL FIELD

The disclosure relates to an electronic device and an operating method. More particularly, the disclosure relates to an electronic device for providing muscular exercise guidance and an operating method thereof.

### BACKGROUND ART

Recently, the development of mobile communication technologies leads to the wide-spread use of a portable or mobile electronic device (e.g., a smart phone, a wearable electronic device, a mobile terminal, or a tablet Personal Computer (PC)), thereby gradually diversifying services providable using the electronic device. The electronic device implemented to be portable or wearable by a user and thus closely related to a user's daily life may be effectively utilized for various services.

A wearable electronic device (e.g., a smart watch, a smart band) may continuously monitor data related to user's biometric data or data related to exercises, sleep, and/or diet and provide a healthcare service. For example, the wearable electronic device may obtain user's biometric data or motion data via one or more sensors, analyze the obtained data, and either store a result of the analysis in conjunction with an application (e.g., a health application, an exercise application) or provide exercise guidance (or coaching) according to the analysis result.

The above information is presented as background information only to assist with an understanding of the disclosure. No determination has been made, and no assertion is made, as to whether any of the above might be applicable as prior art with regard to the disclosure.

### DISCLOSURE

### Technical Solution

Aspects of the disclosure are to address at least the above-mentioned problems and/or disadvantages and to provide at least the advantages described below. Accordingly, an aspect of the disclosure is to provide an electronic device for providing muscular exercise guidance and an operating method thereof.

Additional aspects will be set forth in part in the description which follows and, in part, will be apparent from the description, or may be learned by practice of the presented embodiments.

In accordance with an aspect of the disclosure, a wearable electronic device is provided. The wearable electronic device includes a biosensor including at least one light emitter and at least one light receiver, the biosensor being configured to sense blood flow of a user wearing the wearable electronic device, communication circuitry, an output interface, memory, comprising one or more storage media, storing instructions, and one or more processors communicatively coupled to the biosensor, the communication circuitry, the output interface, and the memory, wherein the instructions, when executed by the one or more processors individually or collectively, cause the wearable electronic device to obtain biometric data related to the blood flow of the user via the biosensor while an application related to a muscular exercise is executed, identify a grip strength change based on a change in the blood flow observed in the biometric data resulting from the muscular exercise performed by the user, create exercise information related to the muscular exercise based on the grip strength change, and provide the user with the exercise information via at least one of the communication circuitry or the output interface.

In accordance with another aspect of the disclosure, a method performed by a wearable electronic device is provided. The method includes obtaining, by the wearable electronic device via a biosensor including at least one light emitter and at least one light receiver, biometric data related to blood flow of a user wearing the wearable electronic device while an application related to a muscular exercise is executed, identifying, by the wearable electronic device, a grip strength change based on a change in the blood flow observed in the biometric data resulting from the muscular exercise performed by the user, creating, by the wearable electronic device, exercise information related to the muscular exercise based on the grip strength change, and providing, by the wearable electronic device, the exercise information to the user.

In accordance with another aspect of the disclosure, one or more non-transitory computer-readable storage media storing one or more computer programs including computer-executable instructions that, when executed by one or more processors of a wearable electronic device individually or collectively, cause the wearable electronic device to perform operations are provided. The operations include obtaining, by the wearable electronic device via a biosensor including at least one light emitter and at least one light receiver, biometric data related to blood flow of a user wearing the wearable electronic device while an application related to a muscular exercise is executed, identifying, by the wearable electronic device, a grip strength change based on a change in the blood flow observed in the biometric data resulting from the muscular exercise performed by the user, creating, by the wearable electronic device, exercise information related to the muscular exercise based on the grip strength change, and providing, by the wearable electronic device, the exercise information to the user.

Other aspects, advantages, and salient features of the disclosure will become apparent to those skilled in the art from the following detailed description, which, taken in conjunction with the annexed drawings, discloses various embodiments of the disclosure.

### DESCRIPTION OF DRAWINGS

The above and other aspects, features, and advantages of certain embodiments of the disclosure will be more apparent from the following description taken in conjunction with the accompanying drawings, in which:

FIG. 1 is a block diagram of an electronic device in a network environment according to an embodiment of the disclosure;

FIG. 2 is a block diagram of an electronic device according to an embodiment of the disclosure;

FIG. 3 is a flowchart illustrating a method of operating an electronic device according to an embodiment of the disclosure;

FIG. 4A is a front perspective view of a wearable electronic device according to an embodiment of the disclosure;

FIG. 4B is a rear perspective view of a wearable electronic device according to an embodiment of the disclosure;

FIG. 5 illustrates an example of a process in which an electronic device provides exercise information according to an embodiment of the disclosure;

FIG. 6 is a flowchart illustrating a method of operating an electronic device according to an embodiment of the disclosure;

FIG. 7A is a graph illustrating an example of a non-grip strength section and grip strength section of sensor data according to an embodiment of the disclosure;

FIG. 7B is a graph illustrating an example of a non-grip strength section of sensor data according to an embodiment of the disclosure;

FIG. 7C is a graph illustrating an example of a grip strength section of sensor data according to an embodiment of the disclosure;

FIG. 8 is a graph illustrating an example of a repetitive grip strength activity section of sensor data according to an embodiment of the disclosure;

FIG. 9A illustrates a graph of sensor data and an example of a user interface, when a first muscular exercise is performed according to an embodiment of the disclosure;

FIG. 9B illustrates a graph of sensor data and an example of a user interface, when a second muscular exercise is performed according to an embodiment of the disclosure;

FIG. 9C illustrates a graph of sensor data and an example of a user interface, when a third muscular exercise is performed according to an embodiment of the disclosure;

FIG. 10 illustrates an operational environment in which a wearable electronic device and an electronic device operate in conjunction with each other according to an embodiment of the disclosure;

FIG. 11 illustrates an example of a user interface for selecting a muscular exercise according to an embodiment of the disclosure;

FIG. 12A illustrates an example of a user interface for first exercise information according to an embodiment of the disclosure;

FIG. 12B illustrates an example of a user interface for second exercise information according to an embodiment of the disclosure; and

FIG. 12C illustrates an example of a user interface for third exercise information according to an embodiment of the disclosure.

The same reference numerals are used to represent the same elements throughout the drawings.

### MODE FOR INVENTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

A user may wear a wearable electronic device on a specific body part, such as a wrist, to perform various muscular exercises, such as dumbbells, bench presses, chin-ups, deadlifts, climbing, pull-ups, or the like. Recently, the wearable electronic device often includes a motion sensor for measuring motion data based on a motion and a Photoplethysmography (PPG) sensor for measuring heart rate data. However, it may be difficult to provide sufficient guidance by using only the motion data or the heart rate data.

In the muscular exercise, grip strength is a key factor necessary for exercise analysis, monitoring, and/or exercise guidance, and may be a representative indicator of physical fitness assessment.

When the muscular exercise guidance is provided without consideration of the grip strength, only limited guidance based on motion data or heart rate data may be available. In this case, it may be difficult to provide overall guidance for the muscular exercise or precise guidance for unit actions constituting the muscular exercise. When the muscular exercise guidance is provided without consideration of the grip strength, according to characteristics of the muscular exercise in which actions of applying or releasing the grip strength are repeated while performing the exercise, inappropriate exercise guidance for a user's exercise posture may result in a poor exercise effect or high risk of injury. In addition, when a user's gesture is recognized during the muscular exercise without consideration of the grip strength, an error may occur in the gesture recognition, which may deteriorate user experience.

An electronic device and an operating method thereof according to various embodiments of the disclosure may provide muscular exercise guidance in consideration of a grip strength factor, thereby increasing accuracy and efficiency of the muscular exercise guidance.

An electronic device and an operating method thereof according to various embodiments of the disclosure may provide appropriate muscular exercise guidance in accordance with characteristics of the muscular exercise in which actions of applying or releasing grip strength are repeated while performing the exercise.

An electronic device and an operating method thereof according to various embodiments of the disclosure may provide muscular exercise guidance in consideration of real-time feedback based on a change in grip strength, thereby preventing injury and increasing exercise effectiveness.

An electronic device and an operating method thereof according to various embodiments of the disclosure may perform gesture recognition in consideration of a grip strength factor while a muscular exercise is performed, thereby preventing an error in the gesture recognition and thus improving user experience.

Technical problems to be solved in the disclosure are not limited to the technical problems mentioned above, and other technical problems not mentioned herein may be clearly understood by those skilled in the art to which the disclosure pertains from the following descriptions.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a wireless fidelity (Wi-Fi™) chip, a Bluetooth™ chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

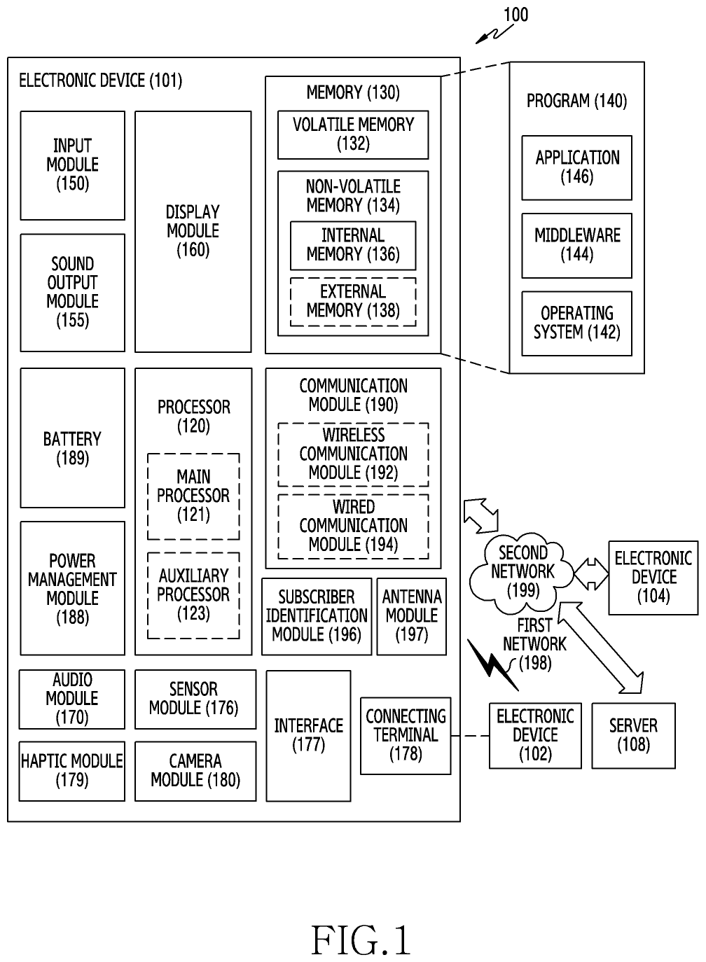

FIG. 1 is a block diagram illustrating an electronic device in a network environment according to an embodiment of the disclosure.

Referring to FIG. 1, an electronic device 101 in a network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., the electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

The connecting terminal 178 may include a connector via which the electronic device 101 may be physically connected with the external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi™) direct, or infrared data association (IrDA)) or the second network 199 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mmWave) band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 gigabits per second (Gbps) or more) for implementing eMBB, loss coverage (e.g., 164 decibels (dB) or less) for implementing mMTC, or U-plane latency (e.g., 0.5 milliseconds (ms) or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices (e.g., the electronic devices 102 and 104 and the server 108). For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

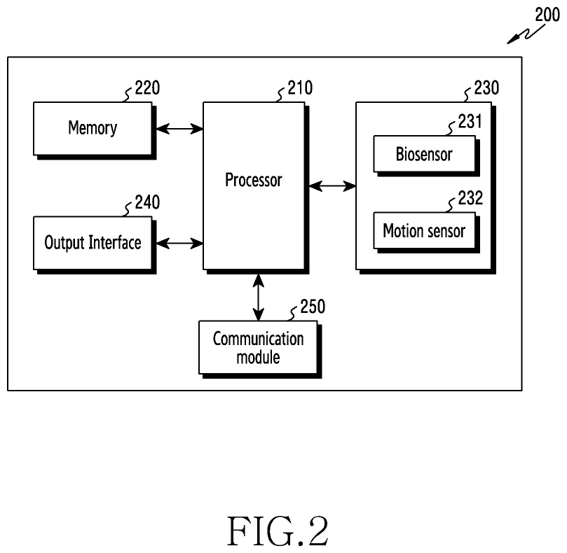

FIG. 2 is a block diagram of an electronic device according to an embodiment of the disclosure.

According to an embodiment, electronic device 200 may be for providing muscular exercise guidance.

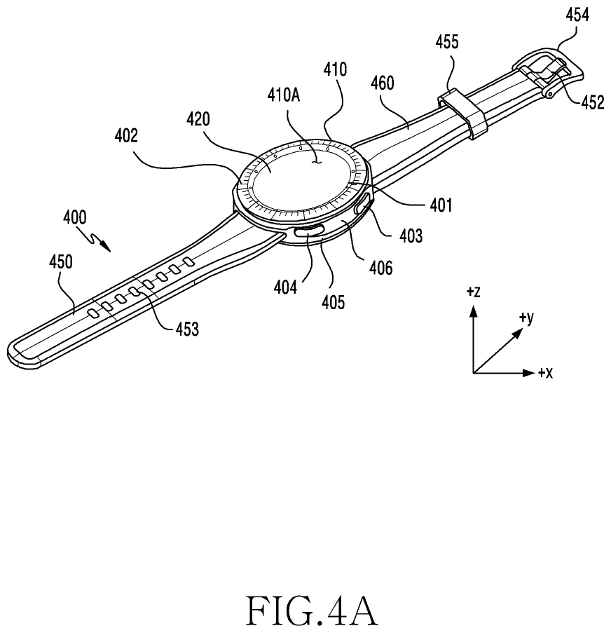

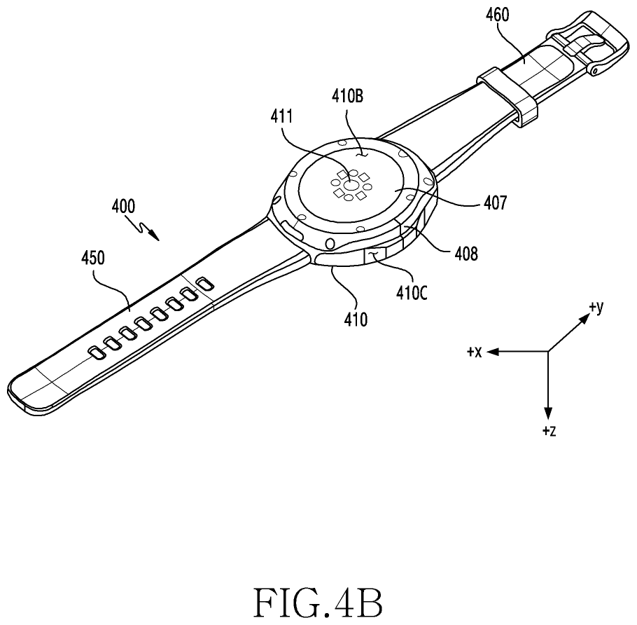

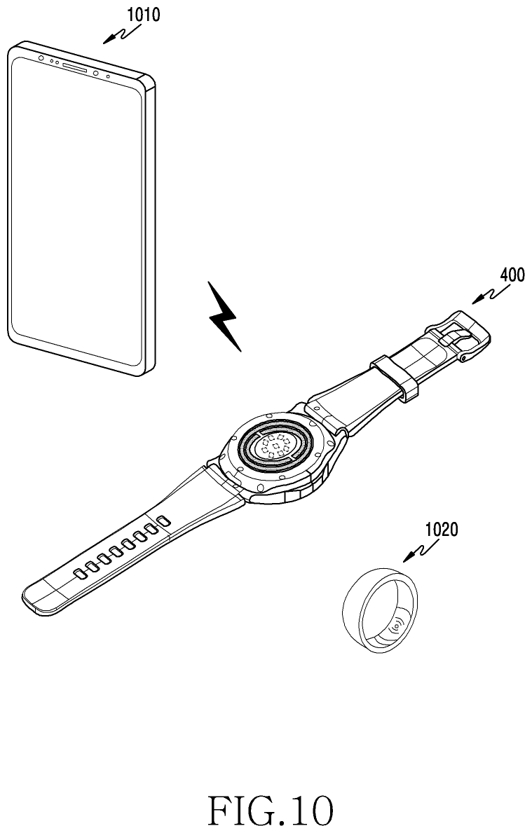

According to an embodiment, electronic device 200 may be implemented as a wearable electronic device of a type that can be worn (or attached). For example, electronic device 200 may be a wearable electronic device 400 (e.g., a smartwatch-type wearable electronic device shown in FIGS. 4A and 4B, smart ring-type wearable electronic device 1020 shown in FIG. 10) that can be worn on a particular body part.

According to an embodiment, electronic device 200 may be implemented as a portable type (e.g., smartphone type) of electronic device (e.g., electronic device 1010 of FIG. 10). For example, electronic device 200 may be an electronic device (e.g., electronic device 1010 of FIG. 10) that is connected via short-range wireless communication with a wearable electronic device (e.g., smart watch, smart band, smart ring, smart patch (or electrode)) worn on a portion of a user's body (e.g., wrist, ankle, finger).

Referring to FIG. 2, an electronic device 200 according to an embodiment (e.g., the electronic device 101 of FIG. 1) may include a processor 210 (e.g., the processor 120 of FIG. 1), memory 220 (e.g., the memory 130 of FIG. 1), a sensor module 230 (e.g., the sensor module 176 of FIG. 1), an output interface 240 (e.g., the sound output module 155, display module 160, audio module 170, and/or haptic module 179 of FIG. 1), and a communication module 250.

According to an embodiment, at least one of the components of the electronic device 200 may be omitted, or other components may be additionally included. The processor 210, memory 220, sensor module 230, output interface 240, and/or communication module 250 included in the electronic device 200 may be electrically and/or operatively coupled to each other to mutually exchange signals (e.g., commands or data).

According to an embodiment, the memory 220 may store instructions. An operation of the processor 210 may be performed by executing the instructions stored in the memory 220. The processor 210 may individually or collectively execute the instructions stored in the memory 220 to perform an arithmetic operation or control the component of the electronic device 200. In the disclosure, it may be understood that the operation of the electronic device 200 is performed when the processor 210 executes the instructions.

According to an embodiment, the processor 210 may include at least one processor including a processing circuitry. The processor 210 may execute and/or control various functions supported in the electronic device 200. The processor 210 may control at least some of the memory 220, the sensor module 230, the output interface 240, and the communication module 250. The processor 210 may execute the instructions stored in the memory 220 of the electronic device 200 and/or code written with programming languages, thereby executing an application and controlling a variety of hardware. For example, the processor 210 may execute an application (e.g., a health application, an exercise application) to provide muscular exercise guidance by using the application. The application executed in the electronic device 200 may operate independently or in conjunction with an external electronic device (e.g., the electronic device 102, the electronic device 104, or the server 108 of FIG. 1).

According to an embodiment, the sensor module 230 may include at least one sensor and/or a sensor circuitry.

According to an embodiment, the sensor module 230 may include a biosensor 231. The biosensor 231 may be for sensing blood flow of a user wearing the electronic device 200 (e.g., the wearable electronic device 400 of FIGS. 4A and 4B or wearable electronic device 1020 of FIG. 10). The biosensor 231 may output biometric data related to the blood flow of the user.

According to an embodiment, the biosensor 231 may include at least one light emitter and at least one light receiver.

According to an embodiment, the biosensor 231 may include a Photoplethysmography (PPG) sensor. Biometric data from the biosensor 231 may be sensor data (PPG data) output from the PPG sensor.

According to an embodiment, the PPG sensor may use light to detect a pulse wave, and may output PPG data corresponding to the pulse wave. The PPG sensor may measure (or detect) a change in a blood flow rate by optically transmitting or reflecting the light to a skin. Since the change in the blood flow rate is caused by a heartbeat (or a heart rate), the PPG sensor may output PPG data (or a PPG signal, e.g., voltage or digital data), based on an amount of light reflected or absorbed due to the change.

According to an embodiment, the PPG sensor may include a light emitting module (or at least one light emitter) and a light receiving module (or at least one light receiver). The light emitting module may output light to the outside in order to create (or obtain) PPG data (or a PPG signal). For example, the light emitting module may include a vertical cavity surface emitting laser (VCSEL), a light emitting diode (LED), a white LED, and/or a white laser. For example, the light emitting module may include various light sources which output light of various wavelength ranges. The light receiving module may receive light output from the light emitting module and reflected from an object (e.g., a user). The light receiving module may convert the received light into an electrical signal. The light emitting module may use the received light to create the PPG data (or the PPG signal). For example, the light emitting module may include an avalanche photodiode (APD), a single photon avalanche diode (SPAD), a photodiode, a photomultiplier tube (PMT), a charge coupled device (CCD), a complementary metal-oxide semiconductor (CMOS) array, and/or a spectrometer.

According to an embodiment, the PPG sensor used as the biosensor 231 may further include a sensor integrated circuit. The sensor integrated circuit may perform at least some of functions of the processor 210 or may operate in conjunction with the processor 210. For example, the sensor integrated circuit may be implemented as a single chip with the light emitting module and/or the light receiving module. For example, the sensor integrated circuit may operate in conjunction with the light emitting module and the light receiving module by being implemented separately from the light emitting module and the light receiving module.

However, a type of the biosensor 231 is not limited to the PPG sensor. For example, the biosensor 231 may be another type of sensor capable of obtaining biometric data related to blood flow (e.g., a blood flow rate, a blood flow state, a heart rate, a heart rate variability, blood pressure, electrocardiogram, blood oxygen concentration, oxygen saturation, and other cardiovascular data). For example, the biosensor 231 may include a near-infrared spectroscopy (NIRS) sensor, an electrocardiography (ECG) sensor, a galvanic skin response (GSR) sensor, a bioelectrical impedance analysis (BIA) sensor, and/or a biomarker sensor which detects a specific substance or component in a body.

According to an embodiment, the sensor module 230 may further include a motion sensor 232. The motion sensor 232 may output motion data based on a user's motion. For example, the motion sensor 232 may include an accelerometer, a gyroscope, a barometer (or an altitude sensor), a gesture sensor, and/or a grip sensor.

According to an embodiment, the output interface 240 may include the display module 160. The display module 160 may provide visual information to the outside (e.g., the user) of the electronic device 200. According to an embodiment, the display module 160, as a touchscreen display, may include a touch circuity configured to detect a touch and/or a sensor circuity (e.g., a pressure sensor) configured to measure strength of force produced by the touch.

According to an embodiment, the communication module 250 may include a communication circuity.

According to an embodiment, the communication module 250 may include a wireless communication module (e.g., the wireless communication module 192 of FIG. 1) (e.g., a cellular communication module, a short-range communication module, or a global navigation satellite system (GNSS) communication module).

According to an embodiment, the communication module 250 may support a short-range wireless communication connection of the electronic device 200 (e.g., the wearable electronic device worn by the user). For example, the communication module 250 may support short-range wireless communication (e.g., Bluetooth™, Bluetooth™ low energy (BLE), wireless fidelity (Wi-Fi™) direct, or infrared data association (IrDA) connection) between the electronic device 200 and an external electronic device (e.g., a smart phone carried by the user while exercising or located near the user).

According to an embodiment, the communication module 250 may support a long-distance wireless communication connection of the electronic device 200. For example, for positioning of the electronic device 200, the communication module 250 may support a GNSS communication connection between a satellite and the electronic device 200 to receive positioning data indicating a current location of the electronic device 200 from the satellite. The communication module 250 may receive the positioning data of the electronic device 200 through long-distance wireless communication and may transfer it to the processor 210 or store it in the memory 220. For example, the communication module 250 may support a long-distance wireless communication (e.g., cellular communication or Internet) connection between an external server (e.g., the server 108 of FIG. 1) and the electronic device 200 to provide the external server with exercise information related to a muscular exercise. For example, the exercise information may be stored in conjunction with a user account managed by the external server.

According to an embodiment, the processor 210 may obtain (e.g., detect, measure, or receive) biometric data (or a biometric signal) related to the user's blood flow via the biosensor 231.

According to an embodiment, the processor 210 may obtain the biometric data while an application (e.g., a health application, an exercise application) related to the muscular exercise is executed. In an embodiment, the processor 210 of the electronic device 200 (e.g., the wearable electronic device 400 or smart watch worn by the user) may obtain biometric data (or a biometric signal) via the biosensor 231 located therein, while the application related to the muscular exercise is executed on the electronic device 200. In an embodiment, the processor 210 of the electronic device 200 (e.g., wearable electronic device 400 or 1020, smart watch, or smart ring worn by the user) may obtain biometric data (or a biometric signal) via the biosensor 231 located therein, while the application related to the muscular exercise is executed on an external electronic device (e.g., electronic device 1010 of FIG. 10) (e.g., a smart phone of the user) coupled through long-distance wireless communication.

According to an embodiment, the processor 210 may activate the biosensor 231 or adjust a measurement cycle (or a measurement frequency) of the biosensor 231 to obtain biometric data. For example, when the application related to the muscular exercise is executed on the electronic device 200 or an external electronic device (e.g., electronic device 1010 of FIG. 10, a user's smart phone) coupled to the electronic device 200 through short-range wireless communication, the processor 210 of the electronic device 200 (e.g., the smart watch or smart ring worn by the user) may activate the biosensor 231 or adjust the measurement cycle of the biosensor 231 from a first cycle to a second cycle shorter than the first cycle to obtain the biometric data.

According to an embodiment, the processor 210 may identify a change in grip strength of the user, based on biometric data from the biosensor 231. The processor 210 may identify the user's grip strength change, based on a change in blood flow observed in the biometric data resulting from the muscular exercise performed (or exercised) by the user. The user's grip strength change may be related to a blood flow change caused by the muscular exercise performed by the user. The muscular exercise performed by the user may lead to a change in the user's blood flow (e.g., volumes, directions, patterns, and fluctuations in blood flow), and the grip strength change may be identified based on the change in the blood flow. The processor 210 may identify the grip strength change, based on biometric data indicating the blood flow change caused by the muscular exercise when the user performs the muscular exercise.

According to an embodiment, the processor 210 may detect an event (or a triggering event) indicating a start of the muscular exercise. In an embodiment, the processor 210 may detect the event indicating the start of the muscular exercise, based on a user input, biometric data from the biosensor 231, and/or motion data from the motion sensor 232. The processor 210 may start to monitor grip strength information indicating the grip strength change in response to the event indicating the start of the muscular exercise.

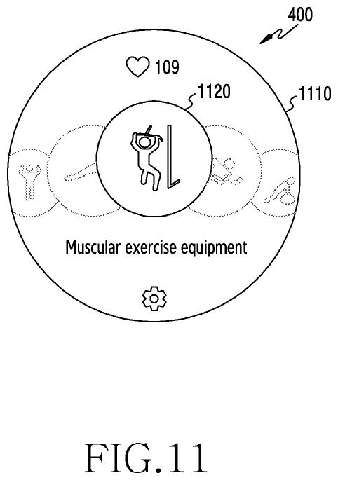

According to an embodiment, the processor 210 may receive the user input for selecting the muscular exercise through a screen (e.g., a touch for a muscular exercise icon 1120 of FIG. 11) of executing an application related to the muscular exercise. The processor 210 may detect the event indicating the start of the muscular exercise, based on the receiving of the user input. The processor 210 may start to monitor the grip strength information indicating the grip strength change in response to the event.

According to an embodiment, the processor 210 may detect the event indicating the start of the muscular exercise, based on a variation in biometric data (or a biometric signal) obtained from the biosensor 231 falling outside a specified threshold range (e.g., a range between a lower threshold and an upper threshold). The processor 210 may start to monitor the grip strength information indicating the grip strength change in response to the event.

According to an embodiment, the processor 210 may obtain motion data (or a motion signal) of the user via the motion sensor 232. The processor 210 may detect an event indicating the start of the muscular exercise, based on detecting of a body motion (e.g., a motion corresponding to a specified body posture for the muscular exercise, a motion of an arm, a motion repeated more than a specific count) of the user from the motion data. The processor 210 may start to monitor the grip strength information indicating the grip strength change in response to the event.

According to an embodiment, the processor 210 may obtain user's motion data via the motion sensor 232. The processor 210 may detect the event indicating the start of the muscular exercise, based on detecting of a stationary state (or a stationary posture, e.g., a state with no positional movement) of the user and a wrist motion (e.g., a local/partial motion) of the user. The processor 210 may start to monitor the grip strength information indicating the grip strength change in response to the event.

According to an embodiment, the processor 210 may monitor the grip strength information, based on the biometric data from the biosensor 231. The grip strength information may indicate a grip strength change resulting from the muscular exercise performed by the user. For example, the grip strength information may include information on whether the grip strength is measured, a start timing of a grip strength activity, a level of the grip strength, an end timing of the grip strength activity, a duration of the grip strength activity, a count of the grip strength activity, a grip strength activity pattern, and an interval between the grip strength activities.

According to an embodiment, the processor 210 may identify a first time section (a non-grip strength section) in which the grip strength is not applied and a second time section (a grip strength section) in which the grip strength is applied, based on the biometric data from the biosensor 231. The processor 210 may filter out first biometric data (e.g., PPG data of a first pattern, normal heart rate data) obtained during the first time section. The processor 210 may monitor second biometric data (e.g., whether the grip strength is measured, a start timing of a grip strength activity, a level of the grip strength, an end timing of the grip strength activity, a duration of the grip strength activity, a count of the grip strength activity, a grip strength activity pattern, and an interval between the grip strength activities), based on second biometric data (e.g., PPG data of a second pattern, abnormal heart rate data, grip strength data) obtained during the second time section.

According to an embodiment, a type of information to be monitored may vary depending on whether the muscular exercise is performed or whether the grip strength is applied. For example, the processor 210 may monitor different types of information by using the single biosensor 231 according to whether the muscular exercise is performed or whether the grip strength is applied. The processor 210 may monitor heart rate information (e.g., heart rate, heart rate variability, oxygen saturation), based on the first biometric data obtained via the biosensor 231 during the first time section in which the grip strength is not applied. The processor 210 may monitor the grip strength information indicating the grip strength change, based on the second biometric data obtained via the biosensor 231 during the second time section in which the grip strength is applied.

According to an embodiment, the processor 210 may determine that a grip strength activity (an activity in which the grip strength is applied or an activity in which the grip strength occurs) has started, when a variation of biometric data (or a biometric signal) obtained from the biosensor 231 exceeds a specified first threshold (e.g., when a slope of the biometric data changes by more than a specified level in a positive direction). The grip strength activity may be a component of the muscular exercise. The processor 210 may determine that the grip strength activity has ended, when the variation of the biometric data (or the biometric signal) obtained from the biosensor 231 exceeds a specified second threshold (e.g., when the slope of the biometric data changes by more than a specified level in a negative direction). The processor 210 may obtain individual grip strength information from the biometric data obtained during a section corresponding to the grip strength activity. The processor 210 may monitor the grip strength information indicating the grip strength change resulting from the muscular exercise performed by a user by collecting the individual grip strength information during a plurality of time sections.

According to an embodiment, the processor 210 may create exercise information related to the muscular exercise, based on the grip strength change (or grip strength information indicating the grip strength change) identified from the biometric data. For example, the exercise information may be for guiding the muscular exercise.

According to an embodiment, exercise information related to the muscular exercise may include information on a risk degree. The processor 210 may determine the risk degree related to a load applied to a wrist due to the muscular exercise, based on the grip strength change (or grip strength information indicating the grip strength change) identified from the biometric data. The processor 210 may provide (e.g., display, output) an indicator for the risk degree (e.g., a risk degree notification message, a warning sound based on the risk degree, a vibration pattern corresponding to the risk degree) as the exercise information related to the muscular exercise via the output interface 240.

According to an embodiment, the exercise information related to the muscular exercise may be for guiding the muscular exercise. For example, the exercise information may include at least part of guidance information on real-time grip strength measurement results, guidance information on muscular exercise analysis results, and guidance information on risk analysis results.

According to an embodiment, the processor 210 may provide the user with exercise information related to the muscular exercise performed by the user. In an embodiment, the processor 210 may provide the user with the exercise information via the communication module 250 and/or the output interface 240. For example, the processor 210 of the electronic device 200 (e.g., the wearable electronic device 400, a smart watch worn by the user) may output a user interface for the exercise information via the output interface 240. For example, the processor 210 of the electronic device 200 (e.g., wearable electronic devices 400 and 1020, a smart watch or smart ring worn by the user) may transmit at least part of the exercise information to an external electronic device (e.g., electronic device 1010 of FIG. 10, a smart phone of the user) via the communication module 250 so that the user interface for the exercise information is output on the external electronic device.

According to an embodiment, the user interface for the exercise information related to the muscular exercise (e.g., a user interface for guiding the muscular exercise) may be implemented as a visual type (e.g., a screen, text), an auditory type (e.g., audio, sound), a tactile type (e.g., vibration), or a hybrid type combining at least some of these types.

According to an embodiment, the processor 210 of the electronic device 200 may provide the exercise information related to the muscular exercise via the output interface 240, for example, the display module 160, audio module 170, sound output module 155, and/or haptic module 179 of FIG. 1. For example, the exercise information may be for guiding the muscular exercise.

In an embodiment, the processor 210 may output a user interface for the exercise information via the output interface 240. For example, the processor 210 may output the user interface of the visual type, the auditory type, the tactile type, or the hybrid type to the user via the output interface 240.

According to an embodiment, the electronic device 200 (e.g., a wearable electronic device worn by the user, the electronic device 101 of FIG. 1, a wearable electronic device 400 of FIG. 10) may provide the exercise information related to the muscular exercise (e.g., the user interface for guiding the muscular exercise) in conjunction with an external electronic device (e.g., a smart phone of the user, the electronic devices 101 and 104 of FIG. 1, electronic device 1010 of FIG. 10). For example, the processor 210 of the electronic device 200 (e.g., wearable electronic devices 400 and 1020 worn by the user) may transmit information on the user interface for guiding the muscular exercise to the external electronic device (e.g., electronic device 1010 of FIG. 10, the smart phone of the user) via the communication module 250, so that at least part (e.g., a screen, a text, voice, vibration) of the user interface is output via the external electronic device. The external electronic device may be in a state of being coupled to the electronic device 200 through short-range wireless communication via the communication module 250.

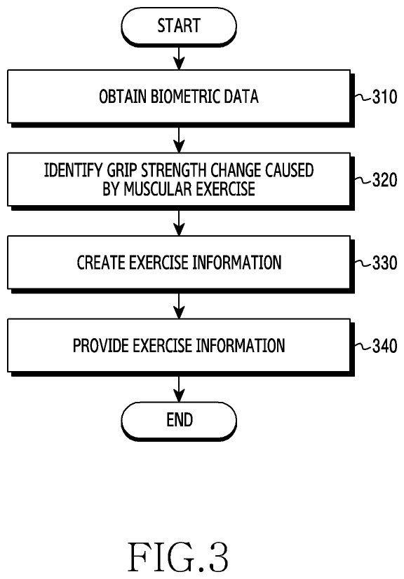

FIG. 3 is a flowchart illustrating a method of operating an electronic device according to an embodiment of the disclosure.

According to an embodiment, the method of FIG. 3 may be for providing muscular exercise guidance. Operations of FIG. 3 may be performed sequentially, but may not be necessarily performed sequentially. For example, orders of the operations may be changed, and at least two operations may be performed in parallel. In some embodiments, some of the operations shown in the drawing may be omitted, or some operations may be combined. Some operations may change in order, or other operations may be added.

According to an embodiment, the electronic device 200 may be a wearable electronic device (e.g., a wearable electronic device 400 of a smart watch type shown in FIGS. 4A and 4B, wearable electronic device 1020 of a smart ring type shown in FIG. 10).

Referring to FIG. 3, the method of operating an electronic device 200 according to an embodiment may include operations 310, 320, 330, and 340.

According to an embodiment, in operation 310, the electronic device 200 (e.g., the wearable electronic device 400) may obtain (e.g., detect, measure, or receive) biometric data (or a biometric signal) related to blood flow of a user via the biosensor 231.

According to an embodiment, the biosensor 231 may include at least one light emitter and at least one light receiver. For example, the biosensor 231 may include a PPG sensor. The biometric data from the biosensor 231 may be sensor data output from the PPG sensor. However, the disclosure is not limited thereto. For example, the biosensor 231 may be another type of sensor capable of obtaining biometric data related to blood flow (e.g., a blood flow rate, a blood flow state, a heart rate, a heart rate variability, blood pressure, electrocardiogram, blood oxygen concentration, oxygen saturation, and other cardiovascular data). For example, the biosensor 231 may include an NIRS sensor, an ECG sensor, a GSR sensor, a BIA sensor, and/or a biomarker sensor.

According to an embodiment, the electronic device 200 (e.g., the wearable electronic device 400) may obtain the biometric data while an application (e.g., a health application, an exercise application) related to the muscular exercise is executed. For example, the wearable electronic device 400 worn by the user may obtain biometric data (or a biometric signal) via the biosensor 231 located therein, while the application related to the muscular exercise is executed on the wearable electronic device 400. In an embodiment, the wearable electronic device worn by the user may obtain biometric data (or a biometric signal) via the biosensor 231 located therein, while the application related to the muscular exercise is executed on an external electronic device (e.g., electronic device 1010 of FIG. 10) coupled through long-distance wireless communication.

According to an embodiment, the electronic device 200 may activate the biosensor 231 or adjust a measurement cycle (or a measurement frequency) of the biosensor 231 to obtain biometric data. For example, the electronic device 200 may activate the biosensor 231 or adjust a measurement cycle of the biosensor 231 from a first cycle to a second cycle shorter than the first cycle to obtain the biometric data, when the application related to the muscular exercise is executed on the electronic device 200 or an external electronic device (e.g., electronic device 1010 of FIG. 10) coupled to the electronic device 200 through short-range wireless communication.

According to an embodiment, in operation 320, the electronic device 200 (e.g., the wearable electronic device 400) may identify a change in grip strength of the user, based on the biometric data obtained through the operation 310. The electronic device 200 may identify the user's grip strength change, based on a change in blood flow observed in the biometric data resulting from the muscular exercise performed (or exercised) by the user. The user's grip strength change may be related to the blood flow change caused by the muscular exercise performed by the user. The muscular exercise performed by the user may lead to a change in the user's blood flow (e.g., volumes, directions, patterns, and fluctuations in blood flow), and the grip strength change may be identified based on the change in the blood flow. The electronic device 200 may identify the grip strength change, based on biometric data indicating the blood flow change caused by the muscular exercise when the user performs the muscular exercise.

According to an embodiment, the operation of identifying the change in user's grip strength (i.e., the operation 320) may include an operation of monitoring grip strength information indicating the grip strength change.

According to an embodiment, the electronic device 200 may detect an event (or a triggering event) indicating a start of the muscular exercise. In an embodiment, the electronic device 200 may detect the event indicating the start of the muscular exercise, based on a user input, biometric data from the biosensor 231, and/or motion data from the motion sensor 232. The electronic device 200 may start to monitor the grip strength information indicating the grip strength change in response to the event indicating the start of the muscular exercise.

According to an embodiment, the electronic device 200 may receive the user input for selecting the muscular exercise through a screen (e.g., a touch for a muscular exercise icon 1120 of FIG. 11) of executing an application related to the muscular exercise. The electronic device 200 may detect the event indicating the start of the muscular exercise, based on the receiving of the user input. The electronic device 200 may start to monitor the grip strength information indicating the grip strength change in response to the event.

According to an embodiment, the electronic device 200 may detect the event indicating the start of the muscular exercise, based on a variation in biometric data (or a biometric signal) obtained from the biosensor 231 falling outside a specified threshold range (e.g., a range between a lower threshold and an upper threshold). The electronic device 200 may start to monitor the grip strength information indicating the grip strength change in response to the event.

According to an embodiment, the electronic device 200 may obtain motion data (or a motion signal) of the user via the motion sensor 232. The electronic device 200 may detect an event indicating the start of the muscular exercise, based on detecting of a body motion (e.g., a motion corresponding to a specified body posture for the muscular exercise, a motion of an arm, a motion repeated more than a specific count) of the user from the motion data. The electronic device 200 may start to monitor the grip strength information indicating the grip strength change in response to the event.

According to an embodiment, the electronic device 200 may obtain user's motion data via the motion sensor 232. The electronic device 200 may detect the event indicating the start of the muscular exercise, based on detecting of a stationary state (or a stationary posture, e.g., a state with no positional movement) of the user and a wrist motion (e.g., a local/partial motion) of the user. The electronic device 200 may start to monitor the grip strength information indicating the grip strength change in response to the event. For example, the electronic device 200 may identify that the user is in the stationary state, based on the motion data from the motion sensor 232 and/or positioning data indicating a current location of the electronic device 200. For example, the stationary state of the user may be a state where there is no positional movement of the user or an overall body motion of the user is less than or equal to a specified level. For example, the stationary state of the user may be a state where the user does not move for a specified period of time after taking a specified body posture (e.g., a standing posture at a specific location, a sitting posture, a lying posture, a basic posture for the muscular exercise). For example, the stationary state of the user may be a state where the user does not move while moving limitedly only within a specific range of motion (e.g., a state where the user moves only a hand, a wrist, or an arm within a specific range of motion while maintaining a specified body posture).

According to an embodiment, in the operation 320, the electronic device 200 may monitor the grip strength information indicating the grip strength change, based on the biometric data obtained through the operation 310. The grip strength information may indicate the grip strength change resulting from the muscular exercise performed by the user. For example, the grip strength information includes information on at least one of whether the grip strength is measured, a start timing of a grip strength activity, a level of the grip strength, an end timing of the grip strength activity, a duration of the grip strength activity, a count of the grip strength activity, a grip strength activity pattern, and an interval between the grip strength activities.

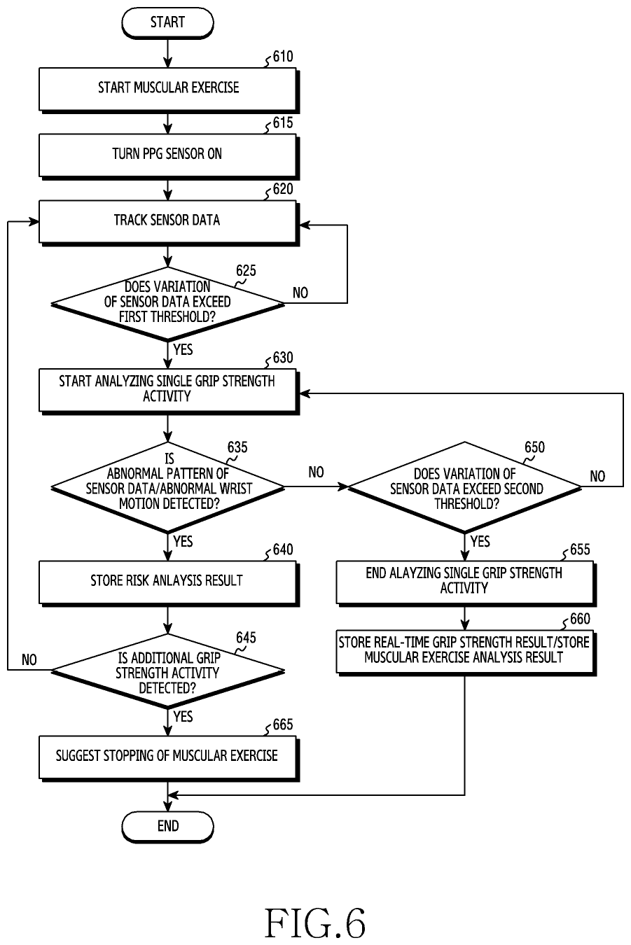

According to an embodiment, the operation of monitoring the grip strength information indicating the grip strength change may include at least some of the operations of FIG. 6 (i.e., operations 625, 630, 635, 640, 645, 650, 655, and 660).

According to an embodiment, the electronic device 200 may identify a first time section (a non-grip strength section) in which the grip strength is not applied and a second time section (a grip strength section) in which the grip strength is applied, based on the biometric data from the biosensor 231. The electronic device 200 may filter out first biometric data (e.g., PPG data of a first pattern, normal heart rate data) obtained during the first time section. The electronic device 200 may monitor second biometric data (e.g., whether the grip strength is measured, a start timing of a grip strength activity, a level of the grip strength, an end timing of the grip strength activity, a duration of the grip strength activity, a count of the grip strength activity, a grip strength activity pattern, and an interval between the grip strength activities), based on second biometric data (e.g., PPG data of a second pattern, abnormal heart rate data, grip strength data) obtained during the second time section.

According to an embodiment, a type of information to be monitored may vary depending on whether the muscular exercise is performed or whether the grip strength is applied. For example, the electronic device 200 may monitor different types of information by using the single biosensor 231 according to whether the muscular exercise is performed or whether the grip strength is applied. The electronic device 200 may monitor heart rate information (e.g., heart rate, heart rate variability, oxygen saturation), based on the first biometric data obtained via the biosensor 231 during the first time section in which the grip strength is not applied. The electronic device 200 may monitor the grip strength information indicating the grip strength change, based on the second biometric data obtained via the biosensor 231 during the second time section in which the grip strength is applied.

According to an embodiment, the electronic device 200 may determine that a grip strength activity (an activity in which the grip strength is applied or an activity in which the grip strength occurs) has started, when a variation of biometric data (or a biometric signal) obtained from the biosensor 231 exceeds a specified first threshold (e.g., when a slope of the biometric data changes by more than a specified level in a positive direction). The grip strength activity may be a component of the muscular exercise. The electronic device 200 may determine that the grip strength activity has ended, when the variation of the biometric data (or the biometric signal) obtained from the biosensor 231 exceeds a specified second threshold (e.g., when the slope of the biometric data changes by more than a specified level in a negative direction). The electronic device 200 may obtain individual grip strength information from the biometric data obtained during a section corresponding to the grip strength activity. The electronic device 200 may monitor the grip strength information indicating the grip strength change resulting from the muscular exercise performed by a user by collecting the individual grip strength information during a plurality of time sections.

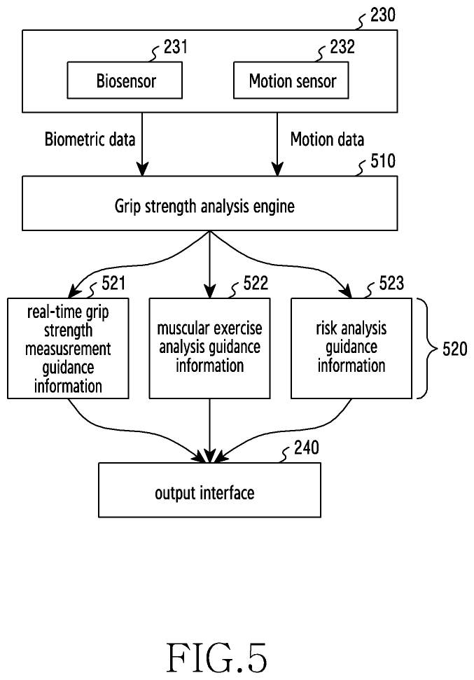

According to an embodiment, in operation 330, the electronic device 200 (e.g., the wearable electronic device 400) may create exercise information (e.g., exercise information 520 of FIG. 5) related to the muscular exercise, based on the grip strength change (or grip strength information indicating the grip strength change) identified in the operation 320. For example, the exercise information may be for guiding the muscular exercise.

According to an embodiment, in operation 340, the electronic device 200 (e.g., the wearable electronic device 400) may provide the user with exercise information (e.g., exercise information 520 of FIG. 5) created in operation 330. In an embodiment, the electronic device 200 may provide the user with the exercise information via the communication module 250 and/or the output interface 240. For example, the electronic device 200 (e.g., the wearable electronic device 400, a smart watch worn by the user) may output a user interface for the exercise information via the output interface 240. For example, the electronic device 200 (e.g., wearable electronic devices 400 and 1020, a smart watch or smart ring worn by the user) may transmit at least part of the exercise information to an external electronic device (e.g., electronic device 1010 of FIG. 10, a smart phone of the user) via the communication module 250 so that the user interface for the exercise information is output on the external electronic device.

According to an embodiment, exercise information related to the muscular exercise may include information on a risk degree. The electronic device 200 may determine the risk degree related to a load applied to a wrist due to the muscular exercise, based on the grip strength change (or grip strength information indicating the grip strength change) identified in the operation 320. The electronic device 200 may provide (e.g., display, output) an indicator for the risk degree (e.g., a risk degree notification message, a warning sound based on the risk degree, a vibration pattern corresponding to the risk degree) as the exercise information related to the muscular exercise via the output interface 240.

According to an embodiment, the exercise information related to the muscular exercise may be for guiding the muscular exercise. For example, the exercise information may include at least part of guidance information (e.g., guidance information 521 of FIG. 5) on real-time grip strength measurement results, guidance information (e.g., guidance information 522 of FIG. 5) on muscular exercise analysis results, and guidance information (e.g., guidance information 523 of FIG. 5) on risk analysis results.

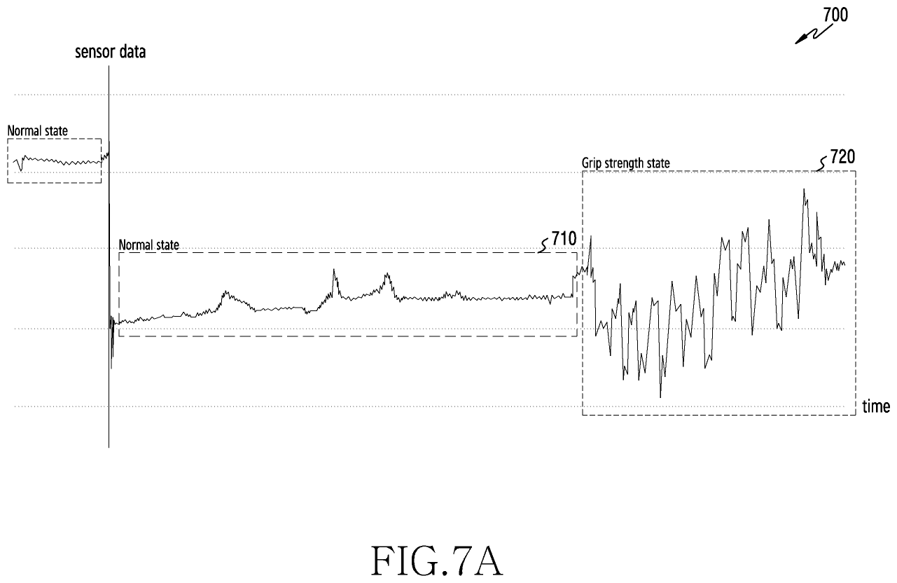

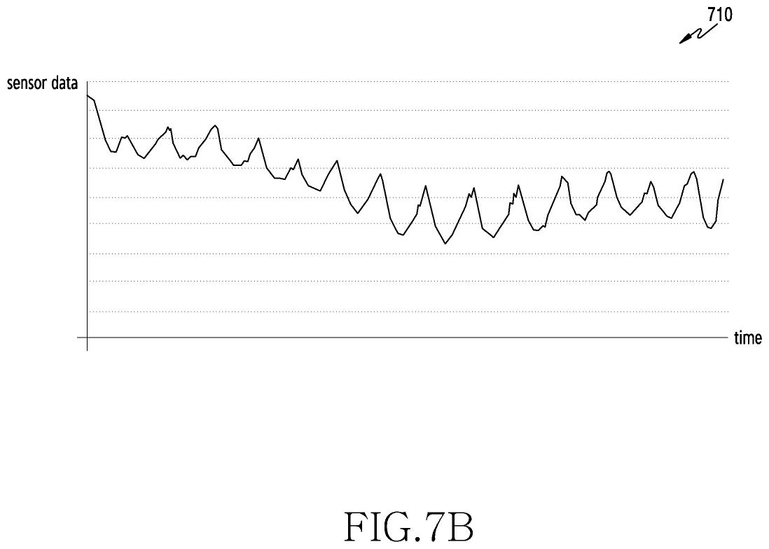

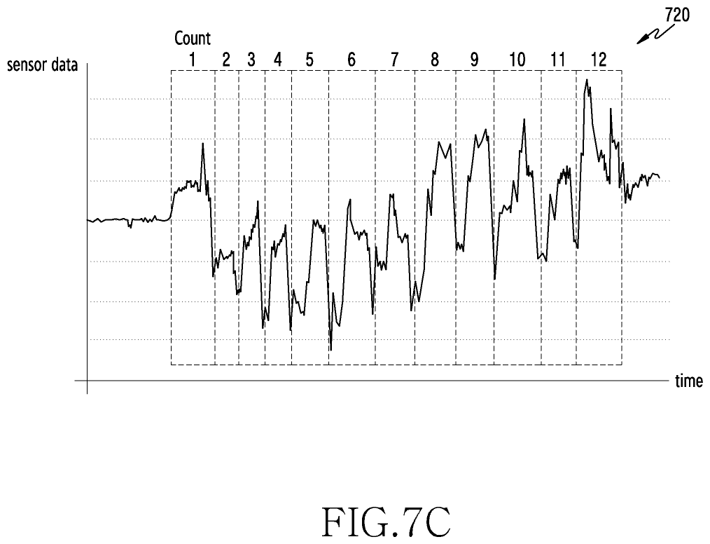

According to an embodiment, the electronic device 200 (e.g., the wearable electronic device 400) may provide guidance information (a grip strength activity start timing, a grip strength activity end timing) on real-time grip measurement results. The electronic device 200 may detect grip strength via the biosensor 231 (e.g., a PPG sensor). For example, when a slope of PPG data (output data of a PPG sensor) obtained during a specified recent time period changes by more than a specified level, the electronic device 200 may determine that the grip strength has occurred. For example, when a slope of a graph indicating PPG data obtained during recent 10 seconds (e.g., graph 700 of FIG. 7A) is below a first threshold (e.g., an absolute value 1) and is close to be horizontal (e.g., non-grip strength section 710 of FIG. 7A), the electronic device 200 may estimate this as a normal state in which no grip strength is applied (or a non-grip strength state). When the slope of the graph indicating the PPG data obtained during recent two seconds is above a second threshold (e.g., an absolute value 10) and is close to be vertical, the electronic device 200 may estimate this as a state in which grip strength is applied (or a grip strength state). When the slope of the PPG data increases significantly in a positive (+) direction compared to the normal state, the electronic device 200 may identify a corresponding timing as a start timing of a grip strength activity. When the slope of the PPG data decreases significantly in a negative (−) direction, the electronic device 200 may identify a corresponding timing as an end timing of the grip strength activity. As such, the electronic device 200 may analyze PPG data obtained during a section estimated as the grip strength state to monitor a real-time grip strength measurement result and/or grip strength information based on the real-time grip strength measurement result. The electronic device 200 may provide guidance information on the real-time grip strength measurement result as the exercise information related to the muscular exercise.

According to an embodiment, the electronic device 200 (e.g., the wearable electronic device 400) may provide guidance information on a muscular exercise analysis result (e.g., an activity/grip strength exercise count, an activity/muscular exercise duration) through grip strength detection as the exercise information related to the muscular exercise.

For example, when the motion sensor 232 is used, it is possible to track a motion of a hand, but it may be difficult to analyze the muscular exercise without the motion of the hand or measure a count of activities constituting the muscular exercise. For example, in the muscular exercise such as a chin-up, an activity (or an action) of lifting a body by applying force to a hand or lowering the body by releasing the force from the hand may be repeated, in a state where the hand (or wrist) is fixed at a specific location or there is no motion of the hand (or wrist). The electronic device 200 may utilize the biosensor 231 (e.g., the PPG sensor) to detect (or measure) grip strength, and may identify a first timing of applying force to the hand in practice during the muscular exercise through the detected grip strength and a second timing of releasing the force from the hand. In general, once the muscular exercise has started, activities (or actions) of continuously applying or releasing the grip strength may be repeated until the end of the muscular exercise. The electronic device 200 may measure an activity/muscular exercise count through grip strength detection using the biosensor 231 (e.g., the PPG sensor). For example, the electronic device 200 may estimate the activity/muscular exercise count through a change in grip strength even for a muscular exercise without a motion of a wrist, such as a chip-up. As such, the electronic device 200 may provide guidance information on a muscular exercise analysis result including the activity/muscular exercise count as the exercise information related to the muscular exercise.

For example, the electronic device 200 may provide guidance focused on a single activity rather than an activity set for multiple activities. The electronic device 200 may utilize the biosensor 231 (e.g., the PPG sensor) to detect (or measure) grip strength, and may identify a first timing of starting applying of force to the hand in practice during the muscular exercise through the detected grip strength and a second timing of releasing the force from the hand. The electronic device 200 may measure a duration (e.g., a duration from the first timing to the second timing) of the singe activity through the detected grip strength. For example, the electronic device 200 may provide guidance information on an activity duration capable of inducing a maximum muscle activity level by using the measured duration. There may be various proper activity durations according to a type of the muscular exercise (or an exercise event). For example, a chin-up may be effectively performed in such a manner that lowering is achieved throughout about 4 seconds and lifting is achieved again throughout about 4 seconds. For example, a push-up may be effectively performed in such a manner that two high-intensity push-ups are performed, followed by three low-intensity push-ups. The electronic device 200 may provide guidance information in an improved activity unit compared to guidance information in an activity set unit (e.g., a guidance message of ‘a few minutes of rest time is recommended.’ or ‘Finish this set in a few minutes.’) while performing the chin-up or the push-up, through grip strength detection. For example, the electronic device 200 may identify an actual start timing of the single activity, and thus may provide activity-unit guidance information (e.g., a guidance message of ‘Hold the current posture for 4 seconds.’ or ‘Two high-intensity push-ups and three low-intensity push-ups have been performed. Rest for 5 seconds and then repeat the same activities.’), indicating an accurate timing as to when to contract or relax muscles. As such, the electronic device 200 may provide guidance information on a muscular exercise analysis result focused on a single activity as the exercise information related to the muscular exercise. Accordingly, precise muscular exercise guidance with more improved quality may be provided to the user.

According to an embodiment, the electronic device 200 (e.g., the wearable electronic device 400) may provide guidance information on a risk analysis result (a risk degree of the muscular exercise, a timing of stopping exercise, suggestion for adjusting an exercise level of difficulty) through the grip strength as the exercise information related to the muscular exercise. For example, the electronic device 200 may provide guidance information for the timing of stopping the exercise or the adjusting of the exercise level of difficulty through a change in grip strength (or a grip strength pattern) detected by the biosensor 231.

According to an embodiment, the electronic device 200 may perform grip strength analysis (or monitor grip strength information) using the biosensor 231 in addition to motion recognition using the motion sensor 232. When only the motion sensor 232 is used, the muscular exercise may be ended only when a condition of performing activities repeatedly a predetermined number of times is satisfied. For example, when a user of the wearable electronic device 400 raises a hand to touch a screen of the wearable electronic device 400 in order to stop the exercise, a gesture of raising the hand may also be recognized (e.g., counted) as an activity count, and accurate motion recognition (e.g., counting of the number of activities) may be difficult. For example, a type of the gesture may include a first gesture in which grip strength occurs (e.g., a gesture of strongly closing and opening a hand, a clench gesture, a gesture in which a wrist shakes and/or an angular change occurs, a gesture of turning or bending the wrist by at least specific angle) and a second gesture in which grip strength does not occur (e.g., moving a hand without grip strength, a simple pinch gesture, a simple finger gesture). The electronic device 200 may perform grip strength analysis to more accurately recognize a user's gesture and a grip strength activity or muscular exercise related to the gesture, regardless of the type.

According to an embodiment, the electronic device 200 may recognize both a first gesture related to the muscular exercise (e.g., the first gesture in which the grip strength occurs) and a second gesture irrelevant to the muscular exercise (e.g., the second gesture in which the grip strength does not occur). Since the electronic device 200 recognizes both the first gesture and the second gesture, it may be possible to measure the activity/muscular exercise count more precisely.

According to an embodiment, the electronic device 200 may detect an excessive weight or an abnormal pattern (e.g., an abnormal pattern of PPG data, an abnormal wrist motion) during the muscular exercise through grip strength analysis. For example, there may be a case where the grip strength is suddenly released or the wrist is excessively bent, and thus a value of sensor data (e.g., PPG data) is temporarily not measured. In this case, the electronic device 200 may provide feedback for a risk degree of the muscular exercise currently being performed by the user, and may provide guidance information which suggests immediate stopping of an activity set. As such, the electronic device 200 may provide guidance information on a risk analysis result, related to the feedback for the risk degree and/or the exercise stop timing, as exercise information related to the muscular exercise. Accordingly, a safer and more positive exercise experience may be provided to the user.

According to an embodiment, the electronic device 200 may detect a gesture based on a user's wrist motion via the motion sensor 232. The user's wrist motion may include a motion of not only the wrist of the user but also a body part corresponding to a hand, finger, and/or arm connected to the wrist. Upon detecting the gesture based on the user's wrist motion via the motion sensor 232, the electronic device 200 may perform or skip monitoring of grip strength information, according to whether grip strength is measured from biometric data. The electronic device 200 may determine whether the grip strength is measured based on the biometric data from the biosensor 231. When the grip strength is measured, the electronic device 200 may determine the detected gesture as the first gesture related to the muscular exercise. When the grip strength is not measured, the electronic device 200 may determine the detected gesture as the second gesture irrelevant to the muscular exercise.

According to an embodiment, when the gesture detected from the motion data is determined as the first gesture related to the muscular exercise, the electronic device 200 may perform (or start) monitoring of grip strength information, based on the biometric data from the biosensor 231. When the gesture detected from the motion data is determined as the second gesture irrelevant to the muscular exercise, the electronic device 200 may skip (or omit) the monitoring of the grip strength information based on the biometric data.

According to an embodiment, when a gesture based on a user's wrist motion is detected via the motion sensor 232, the electronic device 200 may perform different functions, depending on whether grip strength is measured from biometric data. When the gesture detected from the motion data is determined as the first gesture (or a first-type gesture) related to the muscular exercise, the electronic device 200 may perform a first function corresponding to the first gesture. When the gesture detected from the motion data is determined as the second gesture (or a second-type gesture) irrelevant to the muscular exercise, the electronic device 200 may perform a second function corresponding to the second gesture. For example, the first function may be a function (e.g., displaying a user interface (UI) of the first application, counting the number of grip strength activities) of a first application (e.g., a foreground application, a health application, an exercise application). For example, the second function may be a function (e.g., displaying a UI of the second application, displaying a home screen, increasing screen brightness) of a second application (e.g., a background application, a home application) different from the first application.

FIG. 4A is a front perspective view of a wearable electronic device according to an embodiment of the disclosure.

FIG. 4B is a rear perspective view of a wearable electronic device according to an embodiment of the disclosure.

The electronic device 200 according to an embodiment may be a wearable electronic device 400 (e.g., a smart watch) worn on a wrist portion of a user.

According to an embodiment, the wearable electronic device 400 may use a biosensor (e.g., the biosensor 231 of FIG. 2) in a sensor module 411 to detect grip strength and/or to perform monitoring (or grip strength analysis) of grip strength information. The wearable electronic device 400 may provide (or output) exercise information related to a muscular exercise. The wearable electronic device 400 may provide (or output) a user interface indicating guidance information based on results of detecting the grip strength and/or monitoring the grip strength information. The wearable electronic device 400 may detect a gesture based on a wrist motion of a user wearing the wearable electronic device 400 via the biosensor (e.g., the biosensor 231 of FIG. 2) and/or motion sensor (e.g., the motion sensor 232 of FIG. 2) in the sensor module 411.

Referring to FIGS. 4A and 4B, a wearable electronic device 400 according to an embodiment may include a housing 410 including a first face (or a front face) 410A, a second face (or a rear face) 410B, and a side face 410C surrounding a space between the first face 410A and the second face 410B, and binding members 450 and 460 coupled to at least part of the housing 410 and configured to detachably bind the wearable electronic device 400 to a user's body part (e.g., a wrist, an ankle, etc.). In another embodiment (not shown), the housing may refer to a construction which constitutes part of the first face 410A, second face 410B, and side face 410C of FIG. 4A. According to an embodiment, the first face 410A may be constructed of a front plate 401 (e.g., a polymer plate or a glass plate including various coating layers) which is at least partially transparent in practice. The second face 410B may be constructed of a rear plate 407 which is opaque in practice. For example, the rear plate 407 may be constructed of coated or colored glass, ceramic, polymer, metal (e.g., aluminum, stainless steel (STS), or magnesium) or a combination of at least two of these materials. The side face 410C may be constructed with a side bezel structure (or a side member) 406 coupled to the front plate 401 and the rear plate 407 and including metal and/or polymer. In some embodiments, the rear plate 407 and the side bezel structure 406 may be constructed integrally and may include the same material (e.g., a metallic material such as aluminum). The binding members 450 and 460 may be constructed of various materials and shapes. Integrated and multiple unit links may be constructed to be mutually movable by using fabric, leather, rubber, urethane, metal, ceramic, or a combination of at least two of the above materials.

According to an embodiment, the wearable electronic device 400 may include at least one or more of a display 420 (e.g., the display module 160 of FIG. 1, the output interface 240 of FIG. 2), audio modules 405 and 408 (e.g., the sound output module 155 and audio module 170 of FIG. 1, the output interface 240 of FIG. 2), the sensor module 411 (e.g., the sensor module 176 of FIG. 1, the sensor module 230 of FIG. 2), and key input devices 402, 403, and 404 (e.g., the input module 150 of FIG. 2). In some embodiments, the wearable electronic device 400 may omit at least one of elements (e.g., the key input devices 402, 403, and 404), or may additionally include other elements (e.g., one of the components of FIG. 1 or 2).

The display 420 may be exposed through, for example, some portions of the front plate 401. The display 420 may have a shape corresponding to a shape of the front plate 401, and may have various shapes such as a circular shape, an elliptical shape, a polygonal shape, or the like. The display 420 may be disposed adjacent to or joined with a touch sensing circuitry, a pressure sensor capable of measuring touch strength (pressure), and/or a fingerprint sensor.

The audio modules 405 and 408 may include a microphone hole 405 and a speaker hole 408. The microphone hole 405 may have a microphone disposed inside thereof to acquire external sound, and in some embodiments, may have a plurality of microphones disposed to sense a sound direction. The speaker hole 408 may be used as an external speaker and a communication receiver. In some embodiments, the speaker hole 408 and the microphone hole 405 may be implemented with one hole, or the speaker (e.g., a piezo speaker) may be included without the speaker hole 408.

The sensor module 411 may detect (or output) an electrical signal or data corresponding to an internal operational state of the wearable electronic device 400 or an external environmental state. The sensor module 411 may include, for example, a biosensor disposed to the second face 410B of the housing 410 and/or a motion sensor disposed inside the housing 410. The sensor module 411 may detect (or output) motion data based on a motion of the wearable electronic device 400 and/or biometric data (e.g., PPG data, near-infrared spectroscopy (NIRS) data, ECG data, galvanic skin response (GSR) data, BIA data, and biomarker data obtained through a user's wrist in contact with the second face 410B of the housing 410) of a user wearing the wearable electronic device 400 or a corresponding biometric signal.

The key input devices 402, 403, and 404 may include a wheel key 402 disposed to the first face 410A of the housing 410 and rotatable in at least one direction, and/or side key buttons 403 and 404 disposed to the side face 410C of the housing 410. The wheel key 402 may have a shape corresponding to the shape of the front plate 401. In another embodiment, the wearable electronic device 400 may not include some or all of the aforementioned key input devices 402, 403, and 404. The key input devices 402, 403, and 404, which are not included, may be implemented on the display 420 in a different form such as a soft key or the like.

The binding members 450 and 460 may be detachably bound to at least some regions of the housing 410. The binding members 450 and 460 may include one or more of a fixing member 452, a fixing member locking hole 453, a band guide member 454, and a band fixing ring 455.

The fixing member 452 may be configured to fix the housing 410 and the binding members 450 and 460 to a user's body part (e.g., a wrist, an ankle, etc.). The fixing member locking hole 453 may fix the housing 410 and the binding members 450 and 460 to the user's body part in accordance with the fixing member 452. The band guide member 454 may be configured to restrict a motion range of the fixing member 452 when the fixing member 452 is locked to the fixing member locking hole 453, so that the binding members 450 and 460 are bound in close contact with the user's body part. The band fixing ring 455 may restrict the motion range of the binding members 450 and 460, in a state where the fixing member 452 and the fixing member locking hole 453 are locked.

FIG. 5 illustrates an example of a process in which an electronic device provides exercise information according to an embodiment of the disclosure.

Referring to FIG. 5, according to an embodiment, an electronic device 200 (e.g., processor 210 or memory 220) may include a grip strength analysis engine 510. The grip strength analysis engine 510 may be for creating and/or providing exercise information 520 related to a muscular exercise. The grip strength analysis engine 510 may be implemented as hardware, software, or firmware. The grip strength analysis engine 510 may not necessarily be implemented as physically distinct hardware. For example, to implement the grip strength analysis engine 510, the processor 210 of the electronic device 200 may execute instructions stored in the memory 220, and may control hardware (e.g., the sensor module 230, output interface 240, and communication module 250 of FIG. 2) associated with operations and/or functions.

According to an embodiment, the sensor module 230 of FIG. 2 may include the biosensor 231 and/or the motion sensor 232.

According to an embodiment, the grip strength analysis engine 510 may obtain (e.g., receive) biometric data (or a biometric signal) from the biosensor 231 and/or motion data (or a motion signal) from the motion sensor 232, as input information. The grip strength analysis engine 510 may monitor grip strength information (or identify a change in grip strength), based on the input information, and may create the exercise information 520, based on a result of the monitoring. For example, the exercise information 520 may be for guiding the muscular exercise. The grip strength analysis engine 510 may provide the user with the exercise information 520 as output information. For example, the grip strength analysis engine 510 may output the exercise information 520 via the output interface 240.

According to an embodiment, the muscular exercise (or anaerobic exercise) may be classified into a first activity (or a first-type activity) which is countable using the motion sensor 232 and a second activity (or a second-type activity) which is uncountable using only the motion sensor 232. For example, the first activity may correspond to a non-grip strength activity in which grip strength does not occur. The second activity may correspond to a grip strength activity in which grip strength occurs.

According to an embodiment, the grip strength analysis engine 510 may monitor (e.g., measure, track) the first activity, based on motion data from the motion sensor 232. For example, when the electronic device 200 is the wearable electronic device 400 of a smart watch type, since the wearable electronic device 400 is characterized of being worn on a wrist, a real-time progress (e.g., the number of times of performing) of the first activity may be monitored (e.g., measured, tracked) by detecting a wrist motion (e.g., an arm motion) via the motion sensor 232 in the wearable electronic device 400. It may be difficult to perform overall monitoring of the muscular exercise including the second activity with motion data alone.

According to an embodiment, the grip strength analysis engine 510 may monitor a real-time progress of the second activity (e.g., the number of times of performing the activity, calory consumption, change in grip strength, grip strength duration, a progress status of a pre-determined set of activities, weight, total exercise time (duration)), based on the biometric data from the biosensor 231, for example, second biometric data obtained during the second time section in which grip strength is applied. Accordingly, overall monitoring (e.g., measuring, tracking) may be possible for the real-time progress of the second activity or the muscular exercise including the second activity.

According to an embodiment, the grip strength analysis engine 510 may perform monitoring and/or analysis by combining motion data (e.g., wrist motion) and biometric data (e.g., second biometric data related to a grip strength activity obtained during the second time section in which grip strength is applied). The grip strength analysis engine 510 may provide the exercise information 520 in consideration of a result of the monitoring and/or analysis, thereby improving accuracy and efficiency of muscular exercise guidance. Accordingly, a safer and more positive exercise experience may be provided to the user.

According to an embodiment, the exercise information 520 may include at least part of guidance information 521 on real-time grip strength measurement results, guidance information 522 on muscular exercise analysis results, and guidance information 523 on risk analysis results.

According to an embodiment, the grip strength analysis engine 510 may create the guidance information 521 on real-time grip strength measurement results, based on a monitoring result for a real-time single grip strength activity during a muscular exercise performed by a user (e.g., individual grip strength information detected from second biometric data obtained during the second time section in which grip strength is applied). For example, the guidance information 521 on real-time grip strength measurement results may include guidance information on at least one of a timing at which grip strength occurs (or a start timing of a grip strength activity), a current grip strength level, an end timing of the grip strength activity, a duration of the grip strength activity, a grip strength activity count (or the number of times of applying grip strength), a grip strength activity pattern, an estimation weight, and a grip strength level to be continued for a maximum muscle activity level for the single grip strength activity.

According to an embodiment, the grip strength analysis engine 510 may create the guidance information 522 on muscular exercise analysis results, based on a result of comprehensively monitoring real-time grip strength activities (e.g., a result of collecting the individual grip strength information multiple times). For example, the guidance information 522 on muscular exercise analysis results may include guidance information on at least one of the number of times of repeating the grip strength activity, an interval between grip strength activities, a progress status of a determined set of activities (or a plurality of grouped grip strength activities), the number of times of performing the activity, calory consumption, and total exercise time.

According to an embodiment, the grip strength analysis engine 510 may create the guidance information 523 on risk analysis results, based on an assessment result of a degree of risk which may occur in the muscular exercise performed by the user.

According to an embodiment, out of biometric data from the biosensor 231 (e.g., the PPG sensor), the grip strength analysis engine 510 may extract first biometric data (e.g., PPG data and normal heart rate data of a first pattern) of a first time section in which normal blood flow (e.g., blood flow in a state where grip strength is not applied) occurs and second biometric data (e.g., PPG data, abnormal heart rate data, and grip strength data of a second pattern) of a second time section in which abnormal blood flow (e.g., blood flow in a state where grip strength is applied) occurs. The first time section may be a section in which grip strength is not applied. The first time section may be a section in which normal blood flow occurs. The second time section may be a section in which grip strength is applied. The second time section may be a section in which abnormal blood flow caused by grip strength occurs.

In an embodiment, the grip strength analysis engine 510 may evaluate a risk degree related to a load applied to a wrist (or a risk degree related to injury, e.g., one of a high level, an intermediary level, and a low level), based on grip strength information detected from the second biometric data of the second time section in which grip strength is applied. In an embodiment, the grip strength analysis engine 510 may evaluate the risk degree, based on a current gesture detected from the first biometric data (e.g., PPG data and heart rate data of the first pattern) of the first time section and current grip strength information detected from the second biometric data (e.g., PPG data and grip strength data of the second pattern) of the second time section in which grip strength is applied. For example, the grip strength analysis engine 510 may confirm a grip strength allowance range of the current gesture from information on a grip strength allowance range for each gesture stored in the memory 220, and may compare the confirmed grip strength allowance range and the current grip strength information. The grip strength analysis engine 510 may evaluate the risk degree, based on the comparison.

According to an embodiment, the grip strength analysis engine 510 may use an artificial intelligence model trained based on machine learning, neural networks, and/or deep learning algorithms. The grip strength analysis engine 510 may use a roll-based model or a learning model trained using an artificial intelligence algorithm to analyze input information (e.g., biometric data, output data) and create output information (e.g., the exercise information 520). The grip strength analysis engine 510 may provide (e.g., display, output) the created output information (e.g., the exercise information 520) to the user via the output interface 240.

FIG. 6 is a flowchart illustrating a method of operating an electronic device according to an embodiment of the disclosure.

According to an embodiment, the electronic device 200 may be implemented as the wearable electronic device 400 of a wearable type. For example, the wearable electronic device 400 may be a wearable electronic device which is wearable on a specific body part (e.g., a wrist) (e.g., a smart watch type). According to an embodiment, the biosensor 231 may be a PPG sensor. Sensor data from the biosensor 231 may be PPG data.

According to an embodiment, the method of FIG. 6 may be performed by the wearable electronic device 400. The method may be for providing muscular exercise guidance. Operations of FIG. 6 may be performed sequentially, but may not necessarily be performed sequentially. For example, orders of the operations may be changed, and at least two operations may be performed in parallel. In some embodiments, some of the operations shown in the drawing may be omitted, or some operations may be combined. Some operations may change in order, or other operations may be added.

At least some of the operations of FIG. 6 may correspond to the operations of FIG. 3 or may be performed in combination of the operations of FIG. 3.

Referring to FIG. 6, an electronic device 200 (e.g., the wearable electronic device 400 of FIG. 4) according to an embodiment may include operations 610, 615, 620, 625, 630, 635, 640, 645, 650, 655, 660, and 665.

In operation 610, the wearable electronic device 400 may detect an event indicating a start of a muscular exercise.

According to an embodiment, the wearable electronic device 400 may receive a user input for selecting the muscular exercise via a user interface (e.g., a touch on a muscular exercise icon 1120 of FIG. 11) displayed on the display 420. Upon receiving the user input, the wearable electronic device 400 may determine that the event indicating the muscular exercise start occurs.

According to an embodiment, the wearable electronic device 400 may obtain motion data of the user via a motion sensor in the sensor module 411. The wearable electronic device 400 may detect a stationary state of the user (e.g., a state where there is no positional movement of the user or an overall body motion of the user is less than or equal to a specified level) and a wrist motion of the user from the motion data. Upon detecting the stationary state of the user and the wrist motion of the user, the electronic device 200 may determine that the event indicating the muscular exercise start occurs.

In operation 615, the wearable electronic device 400 may activate (or turn-on) a PPG sensor in the sensor module 411.

In operation 620, the wearable electronic device 400 may obtain the PPG data, which is sensor data, from the activated PPG sensor. The wearable electronic device 400 may track (or monitor) the PPG data.

In operation 625, the wearable electronic device 400 may determine whether a variation (e.g., a change level, a change rate, a change range, an increase/decrease rate, a deviation) of sensor data (or PPG data) obtained through the operation 620 exceeds a specified first threshold. For example, the wearable electronic device 400 may determine (or detect) whether a slope of the PPG data has changed significantly (or more than a specified level) in a positive (+) direction. For example, when a user wearing the wearable electronic device 400 starts the muscular exercise and then applies grip strength, first PPG data of a first time section in which normal blood flow occurs (or the grip strength is not applied) may be converted to second PPG data of a second time section in which abnormal blood flow occurs (or the grip strength is applied). The wearable electronic device 400 may detect a start timing of the second time section. The start timing may be a start timing of a grip strength activity (an individual grip strength activity).

When the determination in the operation 625 indicates that the variation of the sensor data exceeds the specified first threshold (e.g., when the slope of the PPG data has changed significantly in the positive (+) direction), the wearable electronic device 400 may proceed to operation 630. In operation 630, the wearable electronic device 400 may start analyzing a single grip activity (or an individual grip activity), based on the sensor data.

According to an embodiment, the wearable electronic device 400 may monitor grip strength information (e.g., whether the grip strength is measured, a start timing of a grip strength activity, a level of the grip strength, an end timing of the grip strength activity, a duration of the grip strength activity, a count of the grip strength activity, a grip strength activity pattern, and an interval between the grip strength activities) from the second PPG data obtained during the second time section in which the abnormal blood flow occurs (or the grip strength is applied).

According to an embodiment, the wearable electronic device 400 may detect PPG data of a first pattern (or a normal heart rate pattern) indicating a heart rate change belonging to a normal range from sensor data of a PPG sensor, and may continuously track a heart rate corresponding to the PPG data of the first pattern. The wearable electronic device 400 may detect PPG data of a second pattern (or an abnormal heart rate pattern, a grip strength pattern) inferable with grip strength from the sensor data of the PPG sensor during the tracking of the heart rate. When the grip strength is applied, PPG data corresponding to sensor data may switch from a first pattern of a first time section to a second pattern of a second time section. In this case, a slope of the PPG data may change more significantly in the second time section than in the first time section. Upon detecting that the slope of the PPG data changes significantly more than a specified level, the wearable electronic device 400 may determine that a single grip strength activity (or an individual grip strength activity) has occurred. Upon determining that the single grip strength activity has occurred, the wearable electronic device 400 may start analyzing grip strength information corresponding to the grip strength activity, based on PPG data of the second time section.

In operation 635, the wearable electronic device 400 may determine (or detect) whether an abnormal pattern of PPG data which is sensor data of the PPG sensor or an abnormal wrist motion is detected. For example, the wearable electronic device 400 may detect an abnormal pattern of PPG data (e.g., e.g., a pattern in which PPG data is interrupted without an abnormal output for a specific period of time) or an abnormal wrist motion (e.g., a motion in which a weight is excessive or a wrist is excessively bent), by using motion data from a motion sensor in the sensor module 411 and/or PPG data from a PPG sensor in the sensor module 411.

When the determination in the operation 635 results in no detection of the abnormal pattern of the PPG data and/or the abnormal wrist motion, the wearable electronic device 400 may proceed to operation 640. In operation 640, the wearable electronic device 400 may evaluate a risk degree related to a load applied to the wrist (or a risk degree related to injury) as a high level, and may store an analysis result in the memory 220.

In operation 645, the wearable electronic device 400 may determine whether an additional grip strength activity is detected. For example, when PPG data of a second pattern (a grip strength pattern) inferable with grip strength from sensor data of the PPG sensor is additionally detected, the wearable electronic device 400 may determine that the additional grip strength activity has occurred.

When the determination in the operation 645 results in detection of the additional grip strength activity, returning to the operation 620, the wearable electronic device 400 may repeat (or continue) tracking (or monitoring) of PPG data which is sensor data of the PPG sensor.

When the determination in the operation 635 results in no detection of the abnormal pattern of the PPG data and/or the abnormal wrist motion, the wearable electronic device 400 may evaluate the risk degree as a low level, and proceed to operation 650.

In operation 650, the wearable electronic device 400 may determine whether a variation (e.g., a change level, a change rate, a change range, an increase/decrease rate, a deviation) of sensor data (or PPG data) obtained from the PPG sensor after the grip strength activity of the operation 625 starts exceeds a specified second threshold. For example, the wearable electronic device 400 may determine (or detect) whether a slope of the PPG data has changed significantly (or more than a specified level) in a negative (−) direction. When the slope of the PPG data has changed significantly in the negative (−) direction (or more than the specified level), the wearable electronic device 400 may determine that a grip strength activity in which a user applies grip strength has stopped, and may proceed to operation 655. For example, when the user wearing the wearable electronic device 400 releases the applied grip strength, second PPG data of the second time section in which abnormal blood flow occurs (or the grip strength is applied) may be converted to first PPG data of the first time section in which normal blood flow occurs (or the grip strength is not applied. The wearable electronic device 400 may detect an end timing of the second time section. The end timing may be an end timing of a grip strength activity (an individual grip strength activity).

In operation 655, the wearable electronic device 400 may end analyzing the single grip strength activity (or the individual grip strength activity) started in the operation 630.

In operation 660, the wearable electronic device 400 may store in the memory 220 a real-time grip strength measurement result and/or a muscular exercise analysis result obtained through the operation 630.

In operation 665, the wearable electronic device 400 may provide a user with a user interface including an indicator which suggests stopping of the muscular exercise. For example, when the determination in the operation 645 results in no detection of the additional grip strength activity, the wearable electronic device 400 may predict (or estimate) a situation in which the muscular exercise has stopped, and may provide (or output) an indicator (e.g., a UI element, a guidance message, a guidance voice, a specified vibration pattern) which suggests the user to stop the muscular exercise.

According to an embodiment, the wearable electronic device 400 may create exercise information (e.g., guidance information on muscular exercise) related to the muscular exercise, based on a risk analysis result stored through the operation 640, a real-time grip strength measurement result stored through the operation 660, and/or a muscular exercise analysis result, and may provide the user with the user interface for the exercise information. For example, the wearable electronic device 400 may display a UI element corresponding to the user interface via the display 420. The wearable electronic device 400 may output a guidance voice corresponding to the user interface via the audio modules 405 and 408 (e.g., a speaker hole). The wearable electronic device 400 may output a specified vibration pattern corresponding to the user interface via a haptic module (e.g., the haptic module 179 of FIG. 1).

FIG. 7A is a graph illustrating an example of a non-grip strength section and grip strength section of sensor data according to an embodiment of the disclosure.

FIG. 7B is a graph illustrating an example of the non-grip strength section of the sensor data according to an embodiment of the disclosure.

FIG. 7C is a graph illustrating an example of the grip strength section of the sensor data according to an embodiment of the disclosure.

According to an embodiment, the electronic device 200 which provides exercise information related to a muscular exercise (e.g., muscular exercise guidance based on grip strength information) may be the wearable electronic device 400 worn on a user's wrist. The biosensor 231 used for monitoring the grip strength information may be a PPG sensor. Biometric data used for monitoring the grip strength information may be PPG data which is sensor data from the PPG sensor.

Referring to FIG. 7A, graph 700 illustrates a trend of changes in PPG data (or a PPG signal) which is an output of the PPG sensor, when the sensor data changes from non-grip strength section 710 to grip strength section 720. The non-grip strength section 710 shows PPG data when a user's state is a normal state (or a non-grip strength state, e.g., a state where no force is applied to a hand and there is no motion). The grip strength section 720 shows PPG data when the user's state is a grip-strength state (e.g., a state where the grip strength is applied).

As shown in the graph 700 of FIG. 7A, the non-grip strength section 710 and the grip strength section 720 are clearly classified in the PPG data.

According to an embodiment, a grip strength activity may not be detected in the non-grip strength section 710. The grip strength activity which is a component of the muscular exercise may be understood as an activity in which the grip strength is applied or an activity in which the grip strength occurs. For example, in a state where the user does not apply the grip strength, there is no external pressure (grip strength) for a blood vessel, and thus a heart rate pattern belonging to a normal range may be detected. The grip strength activity may be detected in the grip strength section 720. For example, in a state where the user applies the grip strength, abnormal blood flow may occur due to the grip strength acting as the external pressure for the blood vessel, and an abnormal heart rate pattern out of a normal range may be detected.

According to an embodiment, during the non-grip strength section 710, the user's state may be the normal state (or the non-grip strength state). The non-grip strength section 710 may be a first time section in which the grip strength is not applied. The non-grip strength section 710 may be the first time section in which normal blood flow occurs. In the non-grip strength section 710, a heart rate belonging to a normal range varies depending on normal blood flow without the grip strength, thereby enabling monitoring of the heart rate information (e.g., a heart rate, a heart rate variability, oxygen saturation).

According to an embodiment, during the grip strength section 720, the user's state may be the grip strength state. The grip strength section 720 may be a second time section in which the grip strength is applied. The grip strength section 720 may be the second time section in which abnormal blood flow occurs. In the grip strength section 720, monitoring of the heart rate information is not possible due to the abnormal blood flow different from the normal blood flow. Instead, the monitoring of the grip strength information may be possible.

The graph of FIG. 7B is enlarged from the non-grip strength section 710 of FIG. 7A. The graph of FIG. 7C is enlarged from the grip strength section 720. The graph of FIG. 7C is an example of PPG data output from the PPG sensor in the wearable electronic device 400 during the grip strength section 720, when a user wearing the wearable electronic device 400 applies force (grip strength) in 12 separate attempts while holding a cylindrical object (e.g., a pull-up bar).

Comparing first PPG data (or a first PPG signal) indicating the non-grip strength section 710 of FIGS. 7A and 7B and second PPG data (or a second PPG signal) indicating the grip strength section 720 of FIGS. 7A and 7C, the PPG data changes relatively more significantly in the grip strength section 720 than in the non-grip strength section 710. When the muscular exercise is performed or the grip strength is applied (the grip strength section 720), a variation of the PPG data may be out of a specified threshold range (e.g., a range of the PPG data of the non-grip strength section 710).

According to an embodiment, the PPG sensor may be a sensor including at least one light emitter and at least one light receiver. For example, the PPG sensor may use an optical signal to obtain PPG data (or a PPG signal) corresponding to a volume of blood flowing in an arterial blood vessel passing through a wrist. When the user does not perform the muscular exercise or does not apply the grip strength (the non-grip strength section 710), since external pressure (grip strength) is not applied to the arterial blood vessel of the user, a relatively large volume of blood may flow in the arterial blood vessel. In this state, the optical signal may be used to enable monitoring of heart rate information. In an embodiment, the electronic device 200 may monitor the heart rate information during the non-grip strength section 710. When the user performs the muscular exercise or applies the grip strength (the grip strength section 720), since external pressure (grip strength) is applied to the arterial blood vessel of the user, a relatively small volume of blood may flow in the arterial blood vessel. In this state, monitoring of the heart rate information may not be possible. In an embodiment, the electronic device 200 may monitor not the heart rate information but the grip strength information during the grip strength section 720.

Referring to FIG. 7B, during non-grip strength section 710, periodic peak signals (or signals having periodicity) based on normal blood flow may be detected from first PPG data (or a first PPG signal). In an embodiment, during the non-grip strength section 710, the electronic device 200 may perform monitoring of heart rate information, based on the first PPG signal including the periodic peak signals.

Referring to FIG. 7C, during grip strength section 720, the periodic peak signals may not be detected due to abnormal blood flow different from the normal blood flow. During the grip strength section 720, second PPG data (or a second PPG signal) out of a specified threshold range (e.g., a range of the first PPG data of the non-grip strength section 710) may be detected. In an embodiment, the electronic device 200 may perform monitoring of grip strength information, based on the second PPG signal.

According to an embodiment, the wearable electronic device 400 may recognize that a situation has occurred in which grip strength is applied at a first timing (a start timing of a grip strength activity) (a situation in which force is applied to a hand) through a PPG data value which changes significantly. The wearable electronic device 400 may recognize that a situation has occurred in which grip strength is released at a second timing (an end timing of the grip strength activity) at which a significant drop occurs in PPG data. The wearable electronic device 400 may use a trend or change in PPG data values at first and second timings to analyze grip strength information (e.g., a start timing of a grip strength activity (a timing at which grip strength is applied), an end timing of the grip strength activity (a timing at which the grip strength disappears), a grip strength level, a duration of the grip strength activity, the number of times the grip strength occurs, an interval).

According to an embodiment, the wearable electronic device 400 may identify a first time section corresponding to the non-grip strength section 710 and a second time section corresponding to the grip strength section 720, based on PPG data (or a PPG signal). The wearable electronic device 400 may filter first PPG data (e.g., PPG data of a first pattern or normal heart rate pattern) obtained during the first time section. The wearable electronic device 400 may monitor and/or analyze grip strength information (e.g., a start timing of a grip strength activity, an end timing of the grip strength activity, a level of the grip strength, a duration of the grip strength activity, a count of the grip strength activity, an interval between the grip strength activities), based on second PPG data (e.g., PPG data of a second pattern, abnormal heart rate pattern, or grip strength pattern) obtained during the second time section.

According to an embodiment, the wearable electronic device 400 may monitor heart rate information (e.g., heart rate, heart rate variability, oxygen saturation), based on first PPG data (or a first PPG signal), during the first time section corresponding to the non-grip strength section 710. The wearable electronic device 400 may monitor and/or analyze grip strength information indicating a grip strength change (whether the grip strength is measured, a start timing of a grip strength activity, a level of the grip strength, an end timing of the grip strength activity, a duration of the grip strength activity, a count of the grip strength activity, a grip strength activity pattern, and an interval between the grip strength activities), based on second PPG data (or a second PPG signal), during the second time section corresponding to the grip strength section 720.

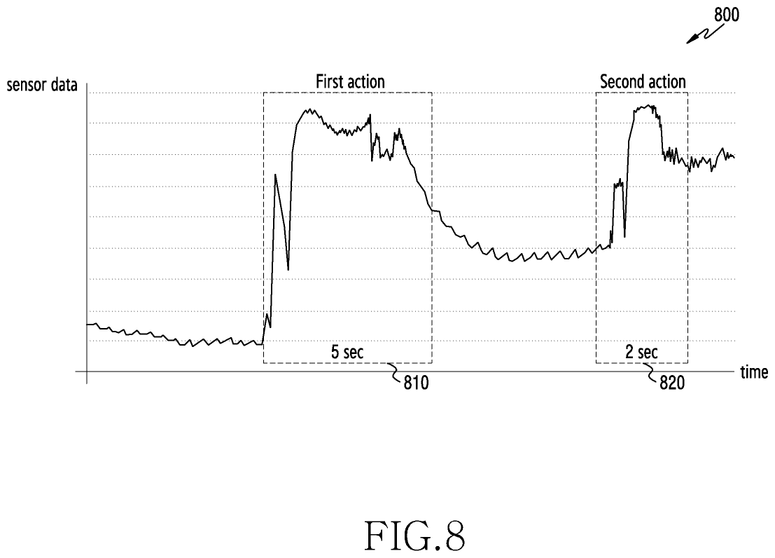

FIG. 8 is a graph illustrating an example of a repetitive grip strength activity section of sensor data according to an embodiment of the disclosure.

Referring to FIG. 8, graph 800 illustrates a trend of changes in PPG data (or a PPG signal), when a user applies grip strength twice (when the user performs two grip strength activities) while wearing the wearable electronic device 400 on a wrist. A first activity section 810 may be a section in which first grip strength is applied (or a first grip strength activity section). A second activity section 820 may be a section in which second grip strength is applied (or a second grip strength activity section). The first activity section 810 indicates a change in PPG data when the grip strength is maintained for a relatively long time (e.g., 5 seconds) in a state where the user holds an object (e.g., a pull-up bar). The second activity section 820 indicates a change in PPG data when the grip strength is maintained for a relatively short time (e.g., 2 seconds) in a state where the user holds the object (e.g., the pull-up bar).

According to an embodiment, the electronic device 200 may recognize multiple grip strength activities separately, based on PPG data shown as in the first activity section 810 and second activity section 820 of FIG. 8. The electronic device 200 may monitor and/or analyze grip strength information such as a start/end timing of each grip strength activity, a level of grip strength, a duration of each grip strength activity, an interval between grip strength activities, a pattern for each grip strength activity, a count of the grip strength activity, or the like.

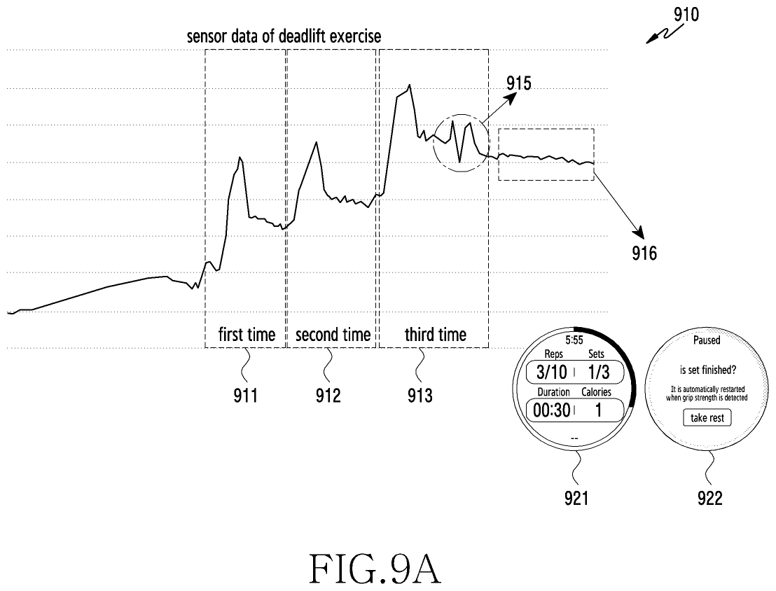

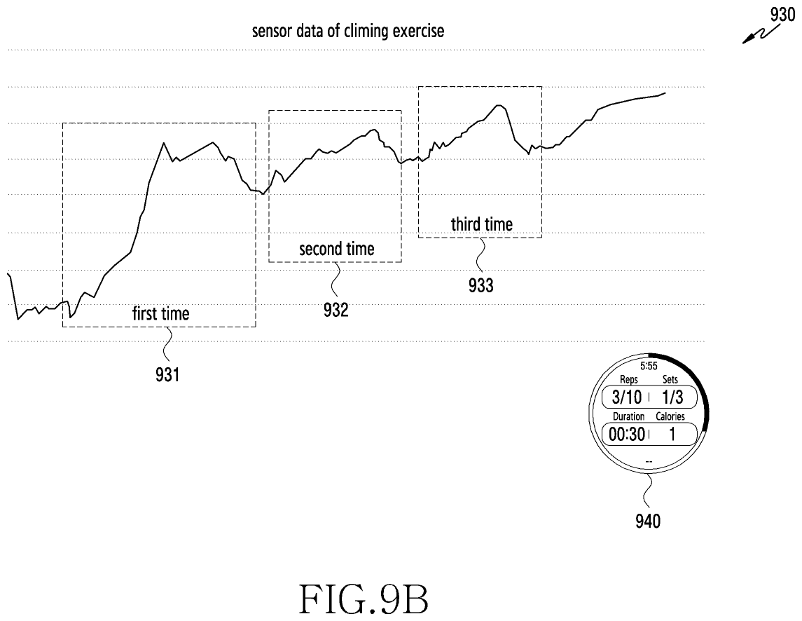

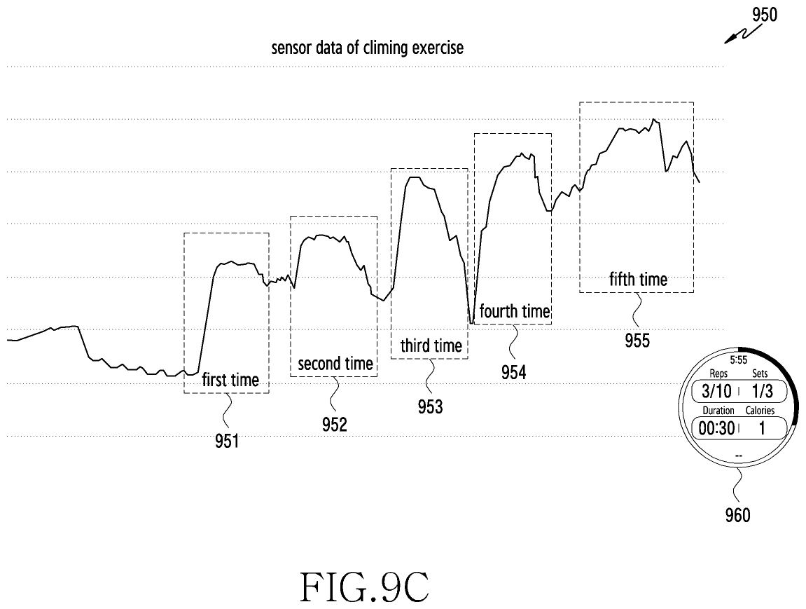

FIGS. 9A, 9B, and 9C illustrate graphs of sensor data of the biosensor 231 according to various muscular exercise types and examples of a user interface displayed on the wearable electronic device 400. The user interface may correspond to an execution screen of an application related to a muscular exercise (e.g., a health application, an exercise application). The user interface may be for providing exercise information related to the muscular exercise (e.g., guidance information on the muscular exercise).

FIG. 9A illustrates a graph of sensor data (e.g., PPG data) and an example of user interfaces displayed on a wearable electronic device, when a first muscular exercise (e.g., a deadlift exercise) is performed according to an embodiment of the disclosure.

According to an embodiment, the deadlift exercise may be a muscular exercise event in which the number of times of activities is countable by using the motion sensor 232.

Referring to FIG. 9A, graph 910 illustrates that, when a user starts the deadlift exercise while holding an object (e.g., a barbell) and then performs three grip strength activities (e.g., lifting the barbell by applying grip strength and lowering the barbell by releasing the grip strength), a wearable electronic device 400 may detect sensor data (e.g., PPG data) including a first activity section 911, a second activity section 912, and a third activity section 913 via the biosensor 231 (e.g., a PPG sensor).

According to an embodiment, the wearable electronic device 400 may detect an abnormal pattern 915 (e.g., an abnormal pattern of PPG data, an abnormal wrist motion) during the deadlift exercise from the sensor data. When the abnormal pattern 915 occurs, the wearable electronic device 400 may use motion data from the motion sensor 232 to perform activity count processing (e.g., adding to the count or excluding from the count). In a subsequent section 916 after the occurrence of the abnormal pattern 915, a change in motion data may be detected in a state where a specified time elapses without detecting grip strength or where the grip strength is not detected. In this case, the wearable electronic device 400 may recognize an exercise stop timing, based on the sensor data (e.g., PPG data) and/or the motion data), and may provide guidance information on the exercise stop timing.

According to an embodiment, the wearable electronic device 400 may display a first user interface 921 including guidance information on grip strength activities detected through the first activity section 911, the second activity section 912, and the third activity section 913. The wearable electronic device 400 may display a second user interface 922 including guidance information related to the exercise stop timing.

FIG. 9B illustrates a graph of sensor data (e.g., PPG data) and a user interface displayed on a wearable electronic device, when a second muscular exercise (e.g., a climbing exercise) is performed according to an embodiment of the disclosure.

Referring to FIG. 9B, according to an embodiment, graph 930 illustrates that, when a user performs the climbing exercise, a wearable electronic device 400 may detect sensor data (e.g., PPG data) including a first activity section 931, a second activity section 932, and a third activity section 933 as shown in FIG. 9B, via the biosensor 231 (e.g., PPG sensor). When the user performs three times in total a grip activity of gripping holds horizontally arranged within a course, sensor data such as the first activity section 931, the second activity section 932, and the third activity section 933 may be detected. The wearable electronic device 400 may detect a first gesture (e.g., a first gesture in which grip strength occurs) related to the muscular exercise (e.g., the climbing exercise) from the sensor data. For example, the first gesture may correspond to an open grip gesture (a grip gesture in which a thumb and four fingers are separated in holding) based on horizontal movement (movement in a horizontal direction).

For example, the wearable electronic device 400 may detect grip strength in each of the activity sections 931, 932, and 933, based on the sensor data (e.g., PPG data), and may provide guidance information on a grip activity and/or grip activity set, based on the detected grip strength. The wearable electronic device 400 may display a user interface 940 including the guidance information. For example, the wearable electronic device 400 may compare grip strength information based on current grip activities and grip strength information based on previous grip activities, and may provide guidance information (e.g., a proper grip count or interval, feedback for a grip duration, a suggestion for adding or excluding grip strength), based on the comparison. The climbing exercise often involves multiple attempts of the same course, and thus such guidance information may be provided to increase accuracy and efficiency of muscular exercise guidance.

FIG. 9C illustrates a graph of sensor data (e.g., PPG data) and an example of a user interface displayed in a wearable electronic device, when a third muscular exercise (e.g., a pull-up exercise) is performed according to an embodiment of the disclosure.

According to an embodiment, the pull-up exercise may be a muscular exercise event in which the number of activities is not countable by using only the motion sensor 232. The wearable electronic device 400 may count the number of times of activities of the pull-up exercise through grip strength detection based on sensor data (e.g., PPG data).

Referring to FIG. 9C, graph 950 illustrates that, when a user starts the pull-up exercise and then performs a grip strength activity (e.g., raising an arm by applying grip strength and lowering the arm by releasing grip strength) five times in total, a wearable electronic device 400 may detect sensor data (e.g., PPG data) including a first activity section 951, a second activity section 952, a third activity section 953, a fourth activity section 954, and a fifth activity section 955 as shown in FIG. 9C via the biosensor 231 (e.g., PPG sensor).

According to an embodiment, the wearable electronic device 400 may display a user interface 960 including guidance information on grip strength activities detected through the first activity section 951, the second activity section 952, the third activity section 953, the fourth activity section 954, and the fifth activity section 955. For example, the guidance information may include information on the number of times of performing the activity, calory consumption, a progress status of a set of activities (or a progress ratio), a total exercise time, or the like.

FIG. 10 illustrates an operational environment in which wearable electronic devices and an electronic device operate in conjunction with each other according to an embodiment of the disclosure.

According to an embodiment, the electronic device 200 of FIG. 2 may be the wearable electronic device 400 or 1020. The electronic device 200 of FIG. 2 may be the wearable electronic device 400 of a smart watch type or the wearable electronic device 1020 of a smart ring type.

Referring to FIG. 10, a wearable electronic device 400 or 1020 according to an embodiment (e.g., a wearable electronic device, smart watch, or smart ring worn by a user) and an electronic device 1010 (e.g., a smart phone of the user) may operate in conjunction with each other. For example, the wearable electronic device 400 may be a wearable electronic device of a smart watch type. The wearable electronic device 1020 may be a wearable electronic device of a smart ring type. The electronic device 1010 may be an electronic device of a smart phone type.

According to an embodiment, the wearable electronic devices 400 and 1020 may be wirelessly coupled to the electronic device 1010. The wearable electronic devices 400 and 1020 may be coupled to the electronic device 1010 through a short-range network supportable by a communication circuitry (e.g., the communication module 250 of FIG. 2), and may mutually transmit and/or receive data. For example, a network (e.g., a short-range network) for establishing a connection between the wearable electronic devices 400 and 1020 and the electronic device 1010 may be appropriately selected. For example, BLE, Wi-Fi™ direct, near field communication (NFC), ultra-wide band (UWB) communication, or infra-red communication may be used, together with Bluetooth™ or instead of Bluetooth™, to establish the connection between the wearable electronic devices 400 and 1020 and the electronic device 1010.

According to an embodiment, the wearable electronic devices 400 and 1020 and/or the electronic device 1010 may provide exercise information related to a muscular exercise (e.g., guidance information on the muscular exercise). The wearable electronic devices 400 and 1020 and/or the electronic device 1010 may provide a user with a user interface which guides the muscular exercise, based on the grip strength information.

According to an embodiment, the wearable electronic device 400 may transmit information on the user interface for guiding the muscular exercise to the electronic device 1010 via the communication module 250, so that at least part (e.g., a screen, a text, voice, vibration) of the user interface is output via the electronic device 1010. The electronic device 1010 may be in a state of being coupled to the wearable electronic devices 400 and 1020 through short-range wireless communication via the communication circuitry (e.g., the communication module 250 of FIG. 2).

FIG. 11 illustrates an example of a user interface for selecting a muscular exercise according to an embodiment of the disclosure.

The user interface may correspond to an execution screen of an application related to the muscular exercise (e.g., a health application, an exercise application). The user interface may be for providing exercise information related to the muscular exercise (e.g., guidance information on the muscular exercise).

According to an embodiment, the wearable electronic device 400 may display a user interface screen 1110 for selecting an exercise type (or an exercise event) via the display 420. The user interface screen 1110 may include a muscular exercise icon 1120. The wearable electronic device 400 may receive a user input for touching the muscular exercise icon 1120. The wearable electronic device 400 may detect an event (or a triggering event) indicating a muscular exercise start, based on receiving of the user input for touching the muscular exercise icon 1120. The wearable electronic device 400 may start to monitor grip strength information indicating a grip strength change, in response to the detecting of the event.

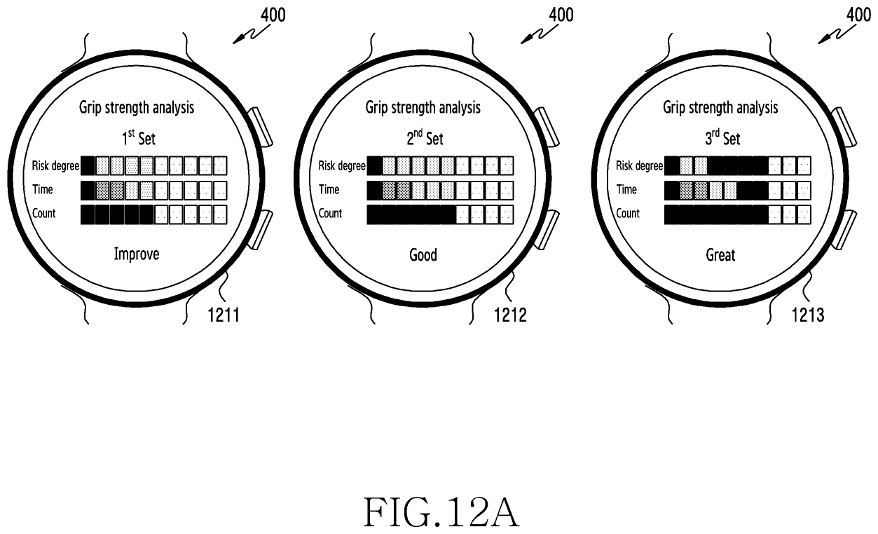

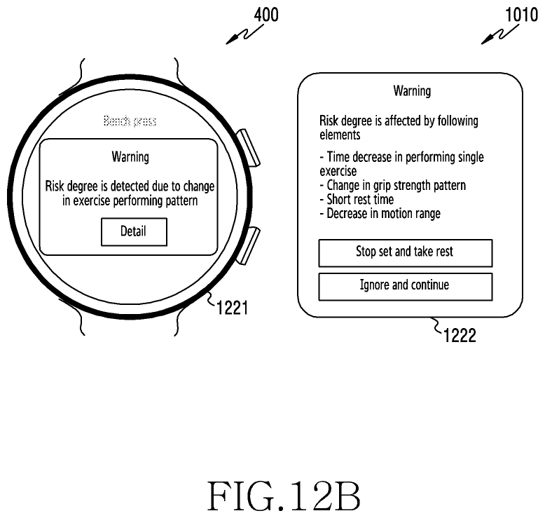

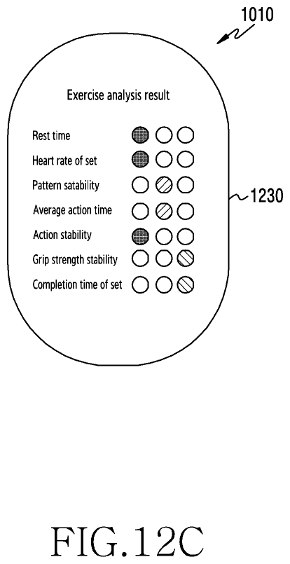

FIG. 12A illustrates an example of a user interface for first exercise information according to an embodiment of the disclosure.

The user interface may be for providing exercise information related to a muscular exercise (e.g., guidance information on the muscular exercise).

Referring to FIG. 12A, according to an embodiment, a wearable electronic device 400 may display user interface screens 1211, 1212, and 1213 on first exercise information. The first exercise information may be for muscular exercise guidance. For example, the first exercise information may be guidance information indicating a real-time measurement result and/or analysis result of activity sets (e.g., a risk degree of each activity set, a grip strength duration, a count, evaluation of exercise) of muscular exercise currently being performed. The wearable electronic device 400 may provide (or output) a user interface (e.g., the user interface screens 1211, 1212, and 1231 of FIG. 12A, a UI element, a guide message, a guide voice, a vibration pattern) for the first exercise information, thereby allowing the user to easily determine on a real-time basis whether the exercise is smoothly in progress.

FIG. 12B illustrates an example of a user interface for second exercise information according to an embodiment of the disclosure.

The user interface may correspond to an execution screen of an application related to a muscular exercise (e.g., a health application, an exercise application).

Referring to FIG. 12B, according to an embodiment, a wearable electronic device 400 may display user interface screens 1221 and 1222 on second exercise information. The second exercise information may be for muscular exercise guidance. For example, the second exercise information is created based on grip strength information detected from biometric data (e.g., PPG data), and may be guidance information on a risk analysis result. The wearable electronic device 400 may provide (or output) a user interface (the user interface screens 1221 and 1222 of FIG. 12A, a UI element, a guide message, a guide voice, a vibration pattern) for the second exercise information. For example, the user interface for the second exercise information may include a UI element indicating a change in an exercise performing pattern (e.g., a grip strength pattern) of the muscular exercise (or an activity, an activity set) currently being performed, a risk degree, or element(s) affecting the risk degree. The user interface on the second exercise information may include an indicator asking for an intention for stopping the muscular exercise (e.g., a menu of ‘stop set and take rest’, ‘ignore and continue’).

According to an embodiment, some parts (e.g., the first user interface screen 1221 of FIG. 12B) of UI elements of the user interface on the second exercise information may be output via the wearable electronic device 400 (e.g., a wearable electronic device worn by a user). The other parts (e.g., the first user interface screen 1222 of FIG. 12B) of the UI elements of the user interface for the second exercise information may be output via the electronic device 1010 (e.g., a smart phone of the user). For example, the wearable electronic device 400 (e.g., the wearable electronic device worn by the user) may output user interfaces synchronized or operating in conjunction with each other, in a state of being coupled through short-range wireless communication.

FIG. 12C illustrates an example of a user interface for third exercise information according to an embodiment of the disclosure.

The user interface may correspond to an execution screen of an application related to a muscular exercise (e.g., a health application, an exercise application).

Referring to FIG. 12C, according to an embodiment, when the muscular exercise ends, the wearable electronic device 400 may display a user interface screen 1230 for the third exercise information. The third exercise information may be for muscular exercise guidance. For example, the third exercise information may be created based on an exercise performing pattern (e.g., a grip strength pattern) and/or grip strength information detected from biometric data (e.g., PPG data) during the muscular exercise. For example, the third exercise information may be guidance information indicating an overall analysis and/or evaluation result for the finished muscular exercise.

According to an embodiment, the user interface screen 1230 may display guidance information on an exercise analysis result related to a degree of risk which may occur while performing the muscular exercise, for example, a rest time, a set heart rate, an exercise performing pattern (e.g., a length of each grip strength activity or each action in a performed set), stability, an average action time, stability of the action, stability of grip strength, and a time taken to complete a set.

According to an embodiment, a wearable electronic device (e.g., the electronic device 101 of FIG. 1, the electronic device 200 of FIG. 2, the wearable electronic device 400 of FIGS. 4A and 4B, the wearable electronic device 1020 of FIG. 10) may include at least one processor (e.g., the processor 120 of FIG. 1, the processor 210 of FIG. 2) including a processing circuitry, a biosensor (e.g., the sensor module 176 of FIG. 1, the biosensor 231 of FIG. 2), a communication circuitry (e.g., the communication module 190 of FIG. 1, the communication module 250 of FIG. 2), an output interface (e.g., the sound output module 155, display module 160, audio module 170, and haptic module 179 of FIG. 1, the output interface 240 of FIG. 2), and memory (e.g., the memory 130 of FIG. 1, the memory 220 of FIG. 2) which stores instructions. The biosensor may be for sensing blood flow of a user wearing the wearable electronic device, and may include at least one light emitter and at least one light receiver. The instructions, when executed individually or collectively by the at least one processor, may cause the wearable electronic device to obtain biometric data related to the blood flow of the user via the biosensor while an application related to a muscular exercise is executed, identify a grip strength change, based on a change in the blood flow observed in the biometric data resulting from the muscular exercise performed by the user, create exercise information related to the muscular exercise, based on the grip strength change, and provide the user with the exercise information via the communication circuitry and/or the output interface.

According to an embodiment, the wearable electronic device may be a wearable electronic device (e.g., the wearable electronic device 400 of FIGS. 4A and 4B) worn on a wrist portion of the user.

According to an embodiment, the wearable electronic device may be a wearable electronic device (e.g., the wearable electronic device 1020 of FIG. 10) worn on a finger of the user.

According to an embodiment, the biosensor may include a PPG sensor.

According to an embodiment, the wearable electronic device may further include a motion sensor.

According to an embodiment, the instructions, when executed individually or collectively by the at least one processor, may cause the wearable electronic device to monitor grip strength information indicating the grip strength change. The grip strength information may include information on at least one of whether the grip strength is measured, a start timing of a grip strength activity, a level of the grip strength, an end timing of the grip strength activity, a duration of the grip strength activity, a count of the grip strength activity, a grip strength activity pattern, and an interval between the grip strength activities.

According to an embodiment, the instructions, when executed individually or collectively by the at least one processor, may cause the wearable electronic device to monitor heart rate information, based on first biometric data obtained via the biosensor, during a first time section in which the grip strength is not applied, and monitor grip strength information indicating the grip strength change, based on second biometric data obtained via the biosensor, during a second time section in which the grip strength is applied.

According to an embodiment, the instructions, when executed individually or collectively by the at least one processor, may cause the wearable electronic device to determine that the grip strength activity has started, when a variation of the biometric data exceeds a specified first threshold, determine that the grip strength activity has ended, when the variation of the biometric data after the grip strength activity has started exceeds a specified second threshold, obtain individual grip strength information from biometric data obtained during a time section corresponding to the grip strength activity, and collect the individual grip strength information to monitor grip strength information indicating the grip strength change.

According to an embodiment, the instructions, when executed individually or collectively by the at least one processor, may cause the wearable electronic device to detect an event indicating a start of the muscular exercise, based on the variation of the biometric data falling outside a specified threshold range, and start to monitor grip strength information indicating the grip strength change, in response to the detecting of the event.

According to an embodiment, the instructions, when executed individually or collectively by the at least one processor, may cause the wearable electronic device to receive a user input for selecting the muscular exercise through an execution screen of the application, detect an event indicating a start of the muscular exercise, based on the receiving of the user input, and start to monitor grip strength information indicating the grip strength change, in response to the detecting of the event.

According to an embodiment, the instructions, when executed individually or collectively by the at least one processor, may cause the wearable electronic device to obtain motion data of the user via a motion sensor of the wearable electronic device, detect an event indicating a start of the muscular exercise, based on detecting of a body motion of the user from the motion data, and start to monitor grip strength information indicating the grip strength change, in response to the detecting of the event.

According to an embodiment, the instructions, when executed individually or collectively by the at least one processor, may cause the wearable electronic device to obtain motion data of the user via a motion sensor of the wearable electronic device, detect an event indicating a start of the muscular exercise, based on detecting of a stationary state of the user and a wrist motion of the user from the motion data, and start to monitor grip strength information indicating the grip strength change, in response to the detecting of the event.

According to an embodiment, the instructions, when executed individually or collectively by the at least one processor, may cause the wearable electronic device to detect a gesture based on a wrist motion of the user via a motion sensor of the wearable electronic device, determine whether grip strength is measured, based on the biometric data, determine the gesture as a first gesture related to the muscular exercise, when the grip strength is measured, and determine the gesture as a second gesture irrelevant to the muscular exercise, when the grip strength is not measured.

According to an embodiment, the instructions, when executed individually or collectively by the at least one processor, may cause the wearable electronic device to monitor grip strength information indicating the grip strength change, when the gesture is determined as the first gesture, and skip the monitoring of the grip strength information, when the gesture is determined as the second gesture.

According to an embodiment, the instructions, when executed individually or collectively by the at least one processor, may cause the wearable electronic device to perform a first function corresponding to the first gesture, when the gesture is determined as the first gesture, and perform a second function corresponding to the second gesture, when the gesture is determined as the second gesture.

According to an embodiment, the instructions, when executed individually or collectively by the at least one processor, may cause the wearable electronic device to determine a risk degree related to a load applied to a wrist due to the muscular exercise, based on grip strength information indicating the grip strength change, and provide an indicator for the risk degree via the output interface.

According to an embodiment, the exercise information includes guidance information for guiding the muscular exercise to the user. The exercise information includes at least portion of guidance information on real-time grip strength measurement results, guidance information on muscular exercise analysis results, and guidance information on risk analysis results.

According to an embodiment, the guidance information for guiding the muscular exercise to the user includes at least portion of information for a time of stopping the muscular exercise and information for adjusting an exercise level of difficulty through the grip strength change.

According to an embodiment, a method of operating a wearable electronic device (e.g., the electronic device 101 of FIG. 1, the electronic device 200 of FIG. 2, the wearable electronic device 400 of FIGS. 4A and 4B, the wearable electronic device 1020 of FIG. 10) may include obtaining biometric data related to blood flow of a user wearing the wearable electronic device, via a biosensor including at least one light emitter and at least one light receiver, while an application related to a muscular exercise is executed, identifying a grip strength change, based on a change in the blood flow observed in the biometric data resulting from the muscular exercise performed by the user, creating exercise information related to the muscular exercise, based on the grip strength change, and providing the exercise information to the user.

According to an embodiment, the identifying of the grip strength change may include monitoring grip strength information indicating the grip strength change. The grip strength information may include information on at least one of whether the grip strength is measured, a start timing of a grip strength activity, a level of the grip strength, an end timing of the grip strength activity, a duration of the grip strength activity, a count of the grip strength activity, a grip strength activity pattern, and an interval between the grip strength activities.

According to an embodiment, the identifying of the grip strength change may include monitoring heart rate information, based on first biometric data obtained via the biosensor, during a first time section in which the grip strength is not applied, and monitoring grip strength information indicating the grip strength change, based on second biometric data obtained via the biosensor, during a second time section in which the grip strength is applied.

According to an embodiment, the identifying of the grip strength change may include determining that the grip strength activity has started, when a variation of the biometric data exceeds a specified first threshold, determining that the grip strength activity has ended, when the variation of the biometric data after the grip strength activity has started exceeds a specified second threshold, obtaining individual grip strength information from biometric data obtained during a time section corresponding to the grip strength activity, and collecting the individual grip strength information to monitor grip strength information indicating the grip strength change.

According to an embodiment, the method may further include detecting an event indicating a start of the muscular exercise, based on the variation of the biometric data falling outside a specified threshold range, and starting to monitor grip strength information indicating the grip strength change, in response to the detecting of the event.

According to an embodiment, the method may further include receiving a user input for selecting the muscular exercise through an execution screen of the application, detecting an event indicating a start of the muscular exercise, based on the receiving of the user input, and starting to monitor grip strength information indicating the grip strength change, in response to the detecting of the event.

According to an embodiment, the method may further include obtaining motion data of the user via a motion sensor of the wearable electronic device, detecting an event indicating a start of the muscular exercise, based on detecting of a body motion of the user from the motion data, and starting to monitor grip strength information indicating the grip strength change, in response to the detecting of the event.

According to an embodiment, the method may further include obtaining motion data of the user via a motion sensor of the wearable electronic device, detecting an event indicating a start of the muscular exercise, based on detecting of a stationary state of the user and a wrist motion of the user from the motion data, and starting to monitor grip strength information indicating the grip strength change, in response to the detecting of the event.

According to an embodiment, the method may further include detecting a gesture based on a wrist motion of the user via a motion sensor of the wearable electronic device, determining whether grip strength is measured, based on the biometric data, determining the gesture as a first gesture related to the muscular exercise, when the grip strength is measured, and determining the gesture as a second gesture irrelevant to the muscular exercise, when the grip strength is not measured.

According to an embodiment, the method may further include monitoring grip strength information indicating the grip strength change, when the gesture is determined as the first gesture, and skipping the monitoring of the grip strength information, when the gesture is determined as the second gesture.

According to an embodiment, the method may further include performing a first function corresponding to the first gesture, when the gesture is determined as the first gesture, and performing a second function corresponding to the second gesture, when the gesture is determined as the second gesture.

According to an embodiment, the method may further include determining a risk degree related to a load applied to a wrist due to the muscular exercise, based on grip strength information indicating the grip strength change, and providing an indicator for the risk degree via the output interface.

According to an embodiment, a storage medium may be a computer readable non-transitory storage medium. The storage medium may have a program recorded to execute a method of operating a wearable electronic device (e.g., the electronic device 101 of FIG. 1, the electronic device 200 of FIG. 2, the wearable electronic device 400 of FIGS. 4A and 4B, the wearable electronic device 1020 of FIG. 10). The storage medium may have a program recorded to execute a method including obtaining biometric data related to blood flow of a user wearing the wearable electronic device, via a biosensor including at least one light emitter and at least one light receiver, while an application related to a muscular exercise is executed, identifying a grip strength change, based on a change in the blood flow observed in the biometric data resulting from the muscular exercise performed by the user, creating exercise information related to the muscular exercise, based on the grip strength change, and providing the exercise information to the user.

According to an embodiment, one or more non-transitory computer-readable storage media storing one or more computer programs including computer-executable instructions that, when executed by one or more processors of a wearable electronic device (e.g., the electronic device 101 of FIG. 1, the electronic device 200 of FIG. 2, the wearable electronic device 400 of FIGS. 4A and 4B, the wearable electronic device 1020 of FIG. 10) individually or collectively, cause the electronic device to perform operations, the operations comprising, obtaining, by the wearable electronic device via a biosensor including at least one light emitter and at least one light receiver, biometric data related to blood flow of a user wearing the wearable electronic device while an application related to a muscular exercise is executed, identifying, by the wearable electronic device, a grip strength change based on a change in the blood flow observed in the biometric data resulting from the muscular exercise performed by the user, creating, by the wearable electronic device, exercise information related to the muscular exercise based on the grip strength change, and providing, by the wearable electronic device, the exercise information to the user.

According to an embodiment, the one or more non-transitory computer-readable storage media of claim 23, the operations further comprising, monitoring, by the wearable electronic device, grip strength information indicating the grip strength change, wherein the grip strength information includes information on at least one of whether the grip strength is measured, a start timing of a grip strength activity, a level of the grip strength, an end timing of the grip strength activity, a duration of the grip strength activity, a count of the grip strength activity, a grip strength activity pattern, or an interval between the grip strength activities.

An electronic device and an operating method thereof according to various embodiments of the disclosure may provide muscular exercise guidance in consideration of a grip strength factor, thereby increasing accuracy and efficiency of the muscular exercise guidance.

An electronic device and an operating method thereof according to various embodiments of the disclosure may provide appropriate muscular exercise guidance in accordance with characteristics of the muscular exercise in which actions of applying or releasing grip strength are repeated while performing the exercise.

An electronic device and an operating method thereof according to various embodiments of the disclosure may provide muscular exercise guidance in consideration of real-time feedback based on a change in grip strength, thereby preventing injury and increasing exercise effectiveness.

An electronic device and an operating method thereof according to various embodiments of the disclosure may perform gesture recognition in consideration of a grip strength factor while a muscular exercise is performed, thereby preventing an error in the gesture recognition and thus improving user experience.

Advantages acquired in the disclosure are not limited to the aforementioned advantages, and other advantages not mentioned herein may be clearly understood by those skilled in the art to which the disclosure pertains from the following descriptions.

Methods based on the embodiments disclosed in the claims and/or specification of the disclosure may be implemented in hardware, software, or a combination of both.

When implemented in software, a computer readable recording medium for storing one or more programs (i.e., software modules) may be provided. The one or more programs stored in the computer readable recording medium are configured for execution performed by one or more processors in the electronic device. The one or more programs include instructions for allowing the electronic device to execute the methods based on the embodiments disclosed in the claims and/or specification of the disclosure.

In the disclosure, a function or operation performed by an electronic device may be performed by at least one processor executing at least one instruction stored in memory. The function or operation of the electronic device mentioned in the disclosure may be performed by one processor executing at least one instruction, or may be performed by a combination of multiple processors executing one instruction. It may be understood that the processor mentioned in this disclosure includes a circuit for controlling other components of the electronic device. For example, the at least one processor may include a Central Processing Unit (CPU), a Micro-Processor Unit (MPU), an Application Processor (AP), a Communication Processor (CP), or a Neural Processing Unit (NPU), a System on Chip (SoC), or an Integrated Circuit (IC), which is configured to execute at least one instruction. The at least one processor may be configured to perform the operation of the electronic device described above.

In the disclosure, programs (e.g., software modules or software) may be stored in random access memory and/or non-volatile memory including flash memory, read only memory (ROM), electrically erasable programmable read only memory (EEPROM), a magnetic disc storage device, a compact disc-ROM (CD-ROM), digital versatile discs (DVDs), other forms of optical storage devices, or a magnetic cassette. Alternatively, the programs may be stored in memory configured in combination of all or some of these storage media. The memory may be constructed of a single storage medium or a combination of a plurality of storage media. The at least one instruction may be stored in the single storage medium, or may be stored in the plurality of storage media in a distributed manner.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it denotes that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101 of FIG. 1). For example, a processor (e.g., the processor 120 of FIG. 1) of the machine (e.g., the electronic device 101 of FIG. 1) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. The term “non-transitory” simply denotes that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device individually or collectively, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A wearable electronic device comprising:
a biosensor including at least one light emitter and at least one light receiver, the biosensor being configured to sense blood flow of a user wearing the wearable electronic device;
communication circuitry;
an output interface;
memory, comprising one or more storage media, storing instructions; and
one or more processors communicatively coupled to the biosensor, the communication circuitry, the output interface, and the memory,
wherein the instructions, when executed by the one or more processors individually or collectively, cause the wearable electronic device to:
obtain biometric data related to the blood flow of the user via the biosensor while an application related to a muscular exercise is executed,
identify a grip strength change based on a change in the blood flow observed in the biometric data resulting from the muscular exercise performed by the user,
create exercise information related to the muscular exercise based on the grip strength change, and
provide the user with the exercise information via at least one of the communication circuitry or the output interface.

2. The wearable electronic device of claim 1,
wherein the instructions, when executed by the one or more processors individually or collectively, further cause the wearable electronic device to:
monitor grip strength information indicating the grip strength change, and
wherein the grip strength information includes information on at least one of whether the grip strength is measured, a start timing of a grip strength activity, a level of the grip strength, an end timing of the grip strength activity, a duration of the grip strength activity, a count of the grip strength activity, a grip strength activity pattern, or an interval between the grip strength activities.

3. The wearable electronic device of claim 1, wherein the instructions, when executed by the one or more processors individually or collectively, further cause the wearable electronic device to:
monitor heart rate information based on first biometric data obtained via the biosensor during a first time section in which the grip strength is not applied; and
monitor grip strength information indicating the grip strength change based on second biometric data obtained via the biosensor during a second time section in which the grip strength is applied.

4. The wearable electronic device of claim 1, wherein the instructions, when executed by the one or more processors individually or collectively, further cause the wearable electronic device to:
determine that a grip strength activity has started when a variation of the biometric data exceeds a specified first threshold;
determine that the grip strength activity has ended when the variation of the biometric data after the grip strength activity has started exceeds a specified second threshold;
obtain individual grip strength information from biometric data obtained during a time section corresponding to the grip strength activity; and
collect the individual grip strength information to monitor grip strength information indicating the grip strength change.

5. The wearable electronic device of claim 1, wherein the instructions, when executed by the one or more processors individually or collectively, further cause the wearable electronic device to:
detect an event indicating a start of the muscular exercise based on a variation of the biometric data falling outside a specified threshold range; and
start to monitor grip strength information indicating the grip strength change in response to the detecting of the event.

6. The wearable electronic device of claim 1, wherein the instructions, when executed by the one or more processors individually or collectively, further cause the wearable electronic device to:
receive a user input selecting the muscular exercise through an execution screen of the application;
detect an event indicating a start of the muscular exercise based on the receiving of the user input; and
start to monitor grip strength information indicating the grip strength change in response to the detecting of the event.

7. The wearable electronic device of claim 1, wherein the instructions, when executed by the one or more processors individually or collectively, further cause the wearable electronic device to:
obtain motion data of the user via a motion sensor of the wearable electronic device;
detect an event indicating a start of the muscular exercise based on detecting body motion of the user from the motion data; and
start to monitor grip strength information indicating the grip strength change in response to the detecting of the event.

8. The wearable electronic device of claim 1, wherein the instructions, when executed by the one or more processors individually or collectively, further cause the wearable electronic device to:
obtain motion data of the user via a motion sensor of the wearable electronic device;
detect an event indicating a start of the muscular exercise based on detecting a stationary state of the user and a wrist motion of the user from the motion data; and
start to monitor grip strength information indicating the grip strength change in response to the detecting of the event.

9. The wearable electronic device of claim 1, wherein the instructions, when executed by the one or more processors individually or collectively, further cause the wearable electronic device to:
detect a gesture based on a wrist motion of the user via a motion sensor of the wearable electronic device;
determine whether grip strength is measured based on the biometric data;
determine the gesture as a first gesture related to the muscular exercise when the grip strength is measured; and
determine the gesture as a second gesture irrelevant to the muscular exercise when the grip strength is not measured.

10. The wearable electronic device of claim 9, wherein the instructions, when executed by the one or more processors individually or collectively, further cause the wearable electronic device to:
monitor grip strength information indicating the grip strength change when the gesture is determined as the first gesture; and
skip the monitoring of the grip strength information when the gesture is determined as the second gesture.

11. The wearable electronic device of claim 9, wherein the instructions, when executed by the one or more processors individually or collectively, further cause the wearable electronic device to:
perform a first function corresponding to the first gesture when the gesture is determined as the first gesture; and
perform a second function corresponding to the second gesture when the gesture is determined as the second gesture.

12. The wearable electronic device of claim 1, wherein the instructions, when executed by the one or more processors individually or collectively, further cause the wearable electronic device to:
determine a risk degree related to a load applied to a wrist of the user due to the muscular exercise based on grip strength information indicating the grip strength change; and
provide an indicator for the risk degree via the output interface.

13. The wearable electronic device of claim 1,
wherein the exercise information includes guidance information for guiding the muscular exercise to the user,
wherein the exercise information includes at least portion of guidance information on real-time grip strength measurement results, guidance information on muscular exercise analysis results, and guidance information on risk analysis results.

14. The wearable electronic device of claim 13, wherein the guidance information for guiding the muscular exercise to the user includes at least portion of information for a time of stopping the muscular exercise and information for adjusting an exercise level of difficulty through the grip strength change.

15. A method performed by a wearable electronic device, the method comprising:
obtaining, by the wearable electronic device via a biosensor including at least one light emitter and at least one light receiver, biometric data related to blood flow of a user wearing the wearable electronic device while an application related to a muscular exercise is executed;
identifying, by the wearable electronic device, a grip strength change based on a change in the blood flow observed in the biometric data resulting from the muscular exercise performed by the user;
creating, by the wearable electronic device, exercise information related to the muscular exercise based on the grip strength change; and
providing, by the wearable electronic device, the exercise information to the user.

16. The method of claim 15,
wherein the identifying of the grip strength change comprises monitoring, by the wearable electronic device, grip strength information indicating the grip strength change, and
wherein the grip strength information includes information on at least one of whether the grip strength is measured, a start timing of a grip strength activity, a level of the grip strength, an end timing of the grip strength activity, a duration of the grip strength activity, a count of the grip strength activity, a grip strength activity pattern, or an interval between the grip strength activities.

17. The method of claim 15, wherein the identifying of the grip strength change comprises:
monitoring, by the wearable electronic device, heart rate information based on first biometric data obtained via the biosensor during a first time section in which the grip strength is not applied; and
monitoring, by the wearable electronic device, grip strength information indicating the grip strength change based on second biometric data obtained via the biosensor during a second time section in which the grip strength is applied.

18. The method of claim 15, wherein the identifying of the grip strength change comprises:
determining, by the wearable electronic device, that a grip strength activity has started based on a variation of the biometric data exceeding a specified first threshold;
determining, by the wearable electronic device, that the grip strength activity has ended based on the variation of the biometric data after the grip strength activity has started exceeding a specified second threshold;
obtaining, by the wearable electronic device, individual grip strength information from biometric data obtained during a time section corresponding to the grip strength activity; and
collecting, by the wearable electronic device, the individual grip strength information to monitor grip strength information indicating the grip strength change.

19. The method of claim 15, further comprising:
detecting, by the wearable electronic device, an event indicating a start of the muscular exercise based on a variation of the biometric data falling outside a specified threshold range; and
starting, by the wearable electronic device, to monitor grip strength information indicating the grip strength change in response to the detecting of the event.

20. One or more non-transitory computer-readable storage media storing one or more computer programs including computer-executable instructions that, when executed by one or more processors of a wearable electronic device individually or collectively, cause the electronic device to perform operations, the operations comprising:
obtaining, by the wearable electronic device via a biosensor including at least one light emitter and at least one light receiver, biometric data related to blood flow of a user wearing the wearable electronic device while an application related to a muscular exercise is executed;
identifying, by the wearable electronic device, a grip strength change based on a change in the blood flow observed in the biometric data resulting from the muscular exercise performed by the user;
creating, by the wearable electronic device, exercise information related to the muscular exercise based on the grip strength change; and
providing, by the wearable electronic device, the exercise information to the user.
